SEQUENCE LISTING (25 sequences)

SEQ ID NO: 1 - DNA, 3328 bp, Aspergillus nidulans
agggccttttcaggggctggttggccaaacctgccgtattttcgattcagggcgggcccttaattctggggaccgatatcccttgaacgcgggggcaatcagttcaaccaacccagaccgctgggcttggtatgagattgcgagcatctatgtcggtgtaccttctgaatgacaatgaatgtattttacttctcgaaaagaacccttgggcactgaattgtgcggagaatgatgccctgattatgatacaactagtccgctccgtcaagccacaagggtctgggcagtccgctataaatcaaaatcgcctgcacgaacagacgaataaccaagaaaacgcccgagcgcgagcgtttcttcttcctctaagccttgcagctggctctgcgtctttgatcaaccctttagctgaatttccccagaacttcagccctctgcatcctgtccttaccgcaactcgttaacctgcgcgacctcgcgcgaccacagccttaggtttcgagatgccatgaaaatcagaaattgaaccccctttccattactatcattctctgcattctgcgagtgatctgtccttcgacgttccttctttccagcgctgcggcgccttcactctcgttgcctacgtttgaccacggtcctacctctcctactgctgattattaggctcctccctacgcctccaatacagggaagtcgccggccatgtttcgtgcacgacgatctcgcatctcgctggtgtttgccgttatatttgtcctcctcatattccactttagccgtctcgcagttacgatcagcctgcaatcttgggtacctccgccgcccgtcgatcaccataatccccctttccccgaccagaacctcaaagatccatacgaaaacgacaatagtgcgaccggcagtggggctcctccgcctgcgttggtagagccagaagaataccaacgaccaccactttacacagattcagatgacagcccaactccgtcaaaagaacgcctggacaccccgagcaatgtcccatctcaggagcctgaatttgatgccgccagacttcagacgggtgcgcagacccaaaataaacatgaagatgatgaggatattgtcccaatttctcactggaagccgatgcccgaacggcatccagtcagtccggaggctttgatcaagctgccaaccgggcaatcaaaggaactcccccaactgcaagctaagttcaaggacgagtcgtcctcggacaagatgcagcggctgcaacaacttgacactatcaagtcggcgttcttacatgcgtggaacggttacaagatctctgccatgggtcatgatgaggttagacctctgcgcggtggtttcaaggacacattcaatggctggggcgcgacccttgtcgacgccttggataccctgtggatcatggatctcaaagaggagttctccatggcagtcgactacgtcaagaaaatcgattttaccaccagcaccaagaaagagattccggtctttgaaaccactattcgctacctaggcgggatgctcggggcctatgatatttcgggacacaaatacgatatacttttggaaaagtctgttgagcttgcggatgtcttgatggacgccttcgacacaccgaaccggatgccaaccctctattataaatggagcccagagtatgcttcagagtttcgccggggggactttaaggctgttctcgccgagcttggctctctctctctcgagttcacgcgtttggcgcagttgaccaaacaggacaagtactacgatgcaattgcacgaatcacaaatgagctcgaaaagtatcaggatttgacaaagcttcccggcttgtggcctctcaacctggacgcatccgggtgcaggcgagttcccggcgtctcgcgagagcctgctgcggctgggcagccagtcagatggtcctctgacgagatcaactcgacgagctcggtatcgtatcgtacaagacaaattcatgagggcggagagcctgtccgtcatgacaatgattcgtttgaaacgggttttcctgtatcagtcgatactcggactcctcccccaaagcaagattgcaccggaggcctcaacgatcagctctcaggcattgacaagttcggactcggagcccttggtgactctacgtacgagtacttaccgaaagagtatatgttgctcggcggtaacaacgaccagtacctcaacatgtatcagaaggccatggacacagtgcgagaatatcttgtttatcagccaatgctcaagaataatcgcgatgtccgcttcttagcgacagttagtatgacaaagagccttgatgcaaaccctccggggcgtaccactttcgcgtacgaaggcactcacctcacctgttttgctggtggtatgcttgccattggcgccaagttgtttgggcttgataaggatctaaagctgggtagtcaactgacggacggctgtgtctgggcatatgaagccacaaagtccggaatcatgccggaagcattccaactggtcccttgtaagaaaggcgagccatgcgaatgggatgaggacgcatactacatggccatggatccttatgccgacaagcggccaatatcacataacaaacgctccgccggccctgaaaaggggaattggcacgtcgtcgccacagccgaatcgtcttcgccccaggaagataaaacacagaaatcaaccactactgagggtcgacacaccggtacaactaccggggcaggcgcgctctcgcacgaggaattcgtcacgggaaaaatcctcaacgaccgactcccgccgggcatgacagggatctcggctcggcagtacctccttcgcccggaggcgatcgagtctgtcttcatcatgttccgcctcacgggcgatccttcctggcgcgaaaagggttggaagatgttccaggctgtcgacaaagccacgaagacggagctggcgaactcggccatttccgacgtaaccgtcgataatccacgcccggtggacagtatggaatcattctggcttgcggagactctgaaatacttctaccttcttttcagcgatccaagcctggtgagccttgacgaatatgtcttgtaagtgatgcttgacttaatcgactgcttgatgctgacttttcccttaggaacaccgaggctcatccgttcaagcgacccaagtactgaagtactaatttaaatgatcttttagcctgtatctatacatggccgctccgctgtagaagcattgataccattaagacagtatcgctgcattcgtgtaccatttgagcttccagaggaacctcttt

SEQ ID NO: 2 - DNA, 2448 bp, Aspergillus nidulans
  misc_feature (1632)..(1632): n is a, g, c, or t/u
atgtttcgtgcacgacgatctcgcatctcgctggtgtttgccgttatatttgtcctcctcatattccactttagccgtctcgcagttacgatcagcctgcaatcttgggtacctccgccgcccgtcgatcaccataatccccctttccccgaccagaacctcaaagatccatacgaaaacgacaatagtgcgaccggcagtggggctcctccgcctgcgttggtagagccagaagaataccaacgaccaccactttacacagattcagatgacagcccaactccgtcaaaagaacgcctggacaccccgagcaatgtcccatctcaggagcctgaatttgatgccgccagacttcagacgggtgcgcagacccaaaataaacatgaagatgatgaggatattgtcccaatttctcactggaagccgatgcccgaacggcatccagtcagtccggaggctttgatcaagctgccaaccgggcaatcaaaggaactcccccaactgcaagctaagttcaaggacgagtcgtcctcggacaagatgcagcggctgcaacaacttgacactatcaagtcggcgttcttacatgcgtggaacggttacaagatctctgccatgggtcatgatgaggttagacctctgcgcggtggtttcaaggacacattcaatggctggggcgcgacccttgtcgacgccttggataccctgtggatcatggatctcaaagaggagttctccatggcagtcgactacgtcaagaaaatcgattttaccaccagcaccaagaaagagattccggtctttgaaaccactattcgctacctaggcgggatgctcggggcctatgatatttcgggacacaaatacgatatacttttggaaaagtctgttgagcttgcggatgtcttgatggacgccttcgacacaccgaaccggatgccaaccctctattataaatggagcccagagtatgcttcagagtttcgccggggggactttaaggctgttctcgccgagcttggctctctctctctcgagttcacgcgtttggcgcagttgaccaaacaggacaagtactacgatgcaattgcacgaatcacaaatgagctcgaaaagtatcaggatttgacaaagcttcccggcttgtggcctctcaacctggacgcatccgggtgcaggcgagttcccggcgtctcgcgagagcctgctgcggctgggcagccagtcagatggtcctctgacgagatcaactcgacgagctcggtatcgtatcgtacaagacaaattcatgagggcggagagcctgtccgtcatgacaatgattcgtttgaaacgggttttcctgtatcagtcgatactcggactcctcccccaaagcaagattgcaccggaggcctcaacgatcagctctcaggcattgacaagttcggactcggagcccttggtgactctacgtacgagtacttaccgaaagagtatatgttgctcggcggtaacaacgaccagtacctcaacatgtatcagaaggccatggacacagtgcgagaatatcttgtttatcagccaatgctcaagaataatcgcgatgtccgcttcttagcgacagttagtatgacaaagagccttgatgcnaaacctccggggcgtaccactttcgcgtacgaaggcactcacctcacctgttttgctggtggtatgcttgccattggcgccaagttgtttgggcttgataaggatctaaagctgggtagtcaactgacggacggctgtgtctgggcatatgaagccacaaagtccggaatcatgccggaagcattccaactggtcccttgtaagaaaggcgagccatgcgaatgggatgaggacgcatactacatggccatggatccttatgccgacaagcggccaatatcacataacaaacgctccgccggccctgaaaaggggaattggcacgtcgtcgccacagccgaatcgtcttcgccccaggaagataaaacacagaaatcaaccactactgagggtcgacacaccggtacaactaccggggcaggcgcgctctcgcacgaggaattcgtcacgggaaaaatcctcaacgaccgactcccgccgggcatgacagggatctcggctcggcagtacctccttcgcccggaggcgatcgagtctgtcttcatcatgttccgcctcacgggcgatccttcctggcgcgaaaagggttggaagatgttccaggctgtcgacaaagccacgaagacggagctggcgaactcggccatttccgacgtaaccgtcgataatccacgcccggtggacagtatggaatcattctggcttgcggagactctgaaatacttctaccttcttttcagcgatccaagcctggtgagccttgaggaatatgtcttgaacaccgaggctcatccgttcaagcgacccaggtactga

SEQ ID NO: 3 - protein, 815 aa, Aspergillus nidulans
MFRARRSRISLVFAVIFVLLIFHFSRLAVTISLQSWVPPPPVDHHNPPFPDQNLKDPTENDNSATGSGAPPPALVEPEETQRPPLTTDSDDSPTPSKERLDTPSNVPSQEPEFDAARLQTGAQTQNKHEDDEDIVPISHWKPMPERHPVSPEALIKLPTGQSKELPQLQAKFKDESSSDKMQRLQQLDTIKSAFLHAWNGTKISAMGHDEVRPLRGGFKDTFNGWGATLVDALDTLWIMDLKEEFSMAVDTVKKIDFTTSTKKEIPVFETTIRTLGGMLGATDISGHKTDILLEKSVELADVLMDAFDTPNRMPTLTTKWSPETASEFRRGDFKAVLAELGSLSLEFTRLAQLTKQDKTTDAIARITNELEKTQDLTKLPGLWPLNLDASGCRRVPGVSREPAAAGQPVRWSSDEINSTSSVSTRTRQIHEGGEPVRHDNDSFETGFPVSVDTRTPPPKQDCTGGLNDQLSGIDKFGLGALGDSTTETLPKETMLLGGNNDQTLNMTQKAMDTVRETLVTQPMLKNNRDVRFLATVSMTKSLDANPPGRTTFATEGTHLTCFAGGMLAIGAKLFGLDKDLKLGSQLTDGCVWATEATKSGIMPEAFQLVPCKKGEPCEWDEDATTMAMDPTADKRPISHNKRSAGPEKGNWHVVATAESSSPQEDKTQKSTTTEGRHTGTTTGAGALSHEEFVTGKILNDRLPPGMTGISARQTLLRPEAIESVFIMFRLTGDPSWREKGWKMFQAVDKATKTELANSAISDVTVDNPRPVDSMESFWLAETLKTFTLLFSDPSLVSLEETVLNTEAHPFKRPRT

SEQ ID NO: 4 - DNA, 2177 bp, Aspergillus nidulans
cggaatgtgcctaaagtggaaggtatgatgatgcccaggatcgcgccccagtcatcaactccatcatgggacggtccttgatcctcaaggcacgaagtggagatcaggtccgtagtgcatatgcatggcccatcagcctgaagcacttccccaagcaaagtcgagactcggacaccgatgatatccctgctgtccctgactgatgcatagtgcatgcccctgcgctggctccccttttcactccgcctggtctccagtctccactcctcaccattgatgtctgcccccgcccgccctccatcctccatcattcttatatctacggactcggtcactcgttatcactagagtccttgtttattcctagtgtttgcattcttacgtgtagttatgcgtacgcttctcgctctcgcggccttcgcgggctttgccgctgctagggtgcccgcctacgccatcacgcgcccggtgatgcgcagtgattctcgcgccgacgctgtcaaggaggccttttcgcatgcctgggacggttactacaactacgcttttcctcatgacgagcttcacccgatttcgaacggttacggagactcgcgaaaccactggggcgcgtcggccgtcgacgctctatcgacggccatcatgatgcgcaacgcgaccatcgtcaaccagatccttgaccatattgctgctgtggactactccaagaccaacgccatggtaagtttgttcgagacgaccatccggtacctcgcgggcatgatttctggatacgacctgctcaaaggccctgcggcggggttggtggacgacagcagggtcgacgtgcttctagagcagtcgcagaacctcgccgaggtgctgaaattcgcgttcgacactccttctggtgtgccgtacaacatgattaacattacttcgggcggcaacgacggcgccacgaccaacgggctggccgtgaccggtaccttggtgctggagtggacgcgtctgtcggacttgactgggaacgacgagtatgcccgcttgagccagagagctgaagactaccttctccacccggagccagcgcagtacgaaccgttccctggattgattggaagcgcagtcaatattgccgacggcaagctcgccaatggtcacatcagctggaatggtggcgcagactcgtactacgagtacctgatcaagatgtacgtctacgatcccgaacgctttggcctctaccgggaccgctgggtcgcagctgccgagtcgagcatcaaccatctggcttcgcacccgtccacccgcccagacgtgactttcttggccacttacaacgaggagcatcagctgggcctgaccagccaacacctgacctgcttcgacggtggaagctttctgcttggtgggacattgctggaccgccaggactttgtcgacttcggccttgaccttgtcgccggctgccacgagacgtacaactcgactctgacgggcatcggccctgagcaattcagctgggaccctaacggtgtgcccgacagccagaaggagctgttcgagcgcgcaggcttctacatcaacagcggccaatacattcttcgtcccgaagtcatcgagagcttctactatgcatggcgcgtcacaggtgatggaacggtacgttcactcagcgctgcttccgtaggaagaccatactgaccgctttagtacctcgaatgggtgtggaacgccttcaccaacatcaacaagtactgccgcactgcgaccggtttcgcggggctggagaacgtcaatgcagcgaacggcggaggccggatcgacaaccaggagagtttcatgttcgcagaggtgctgaagtattcgtttttgacttttgctcctggtacgtttccctagttctggttcacctgtggagaatattactgactgcagcagaggacgactggcaggtgcagaagggcagtggaaatacgtttgtttataacaccgaggcgcacccgtttaaggtgtatacgcctcagtagatagtacatattgtgctctagcgtactgcatgcataacctaaatggcatcacctacttactgactactctactgacaagcagttgcctttatatgctcttaaggagcttgggcggtaaatcgtaattcactctaaactccgcgtgctgttcggggaaggctgggc

SEQ ID NO: 5 - DNA, 1515 bp, Aspergillus nidulans
atgcgtacgcttctcgctctcgcggccttcgcgggctttgccgctgctagggtgcccgcctacgccatcacgcgcccggtgatgcgcagtgattctcgcgccgacgctgtcaaggaggccttttcgcatgcctgggacggttactacaactacgcttttcctcatgacgagcttcacccgatttcgaacggttacggagactcgcgaaaccactggggcgcgtcggccgtcgacgctctatcgacggccatcatgatgcgcaacgcgaccatcgtcaaccagatccttgaccatattgctgctgtggactactccaagaccaacgccatggtaagtttgttcgagacgaccatccggtacctcgcgggcatgatttctggatacgacctgctcaaaggccctgcggcggggttggtggacgacagcagggtcgacgtgcttctagagcagtcgcagaacctcgccgaggtgctgaaattcgcgttcgacactccttctggtgtgccgtacaacatgattaacattacttcgggcggcaacgacggcgccacgaccaacgggctggccgtgaccggtaccttggtgctggagtggacgcgtctgtcggacttgactgggaacgacgagtatgcccgcttgagccagagagctgaagactaccttctccacccggagccagcgcagtacgaaccgttccctggattgattggaagcgcagtcaatattgccgacggcaagctcgccaatggtcacatcagctggaatggtggcgcagactcgtactacgagtacctgatcaagatgtacgtctacgatcccgaacgctttggcctctaccgggaccgctgggtcgcagctgccgagtcgagcatcaaccatctggcttcgcacccgtccacccgcccagacgtgactttcttggccacttacaacgaggagcatcagctgggcctgaccagccaacacctgacctgcttcgacggtggaagctttctgcttggtgggacattgctggaccgccaggactttgtcgacttcggccttgaccttgtcgccggctgccacgagacgtacaactcgactctgacgggcatcggccctgagcaattcagctgggaccctaacggtgtgcccgacagccagaaggagctgttcgagcgcgcaggcttctacatcaacagcggccaatacattcttcgtcccgaagtcatcgagagcttctactatgcatggcgcgtcacaggtgatggaacgtacctcgaatgggtgtggaacgccttcaccaacatcaacaagtactgccgcactgcgaccggtttcgcggggctggagaacgtcaatgcagcgaacggcggaggccggatcgacaaccaggagagtttcatgttcgcagaggtgctgaagtattcgtttttgacttttgctcctgaggacgactggcaggtgcagaagggcagtggaaatacgtttgtttataacaccgaggcgcacccgtttaaggtgtatacgcctcag

SEQ ID NO: 6 - protein, 505 aa, Aspergillus nidulans
MRTLLALAAFAGFAAARVPATAITRPVMRSDSRADAVKEAFSHAWDGTTNTAFPHDELHPISNGTGDSRNHWGASAVDALSTAIMMRNATIVNQILDHIAAVDTSKTNAMVSLFETTIRTLAGMISGTDLLKGPAAGLVDDSRVDVLLEQSQNLAEVLKFAFDTPSGVPTNMINITSGGNDGATTNGLAVTGTLVLEWTRLSDLTGNDETARLSQRAEDTLLHPEPAQTEPFPGLIGSAVNIADGKLANGHISWNGGADSTTETLIKMTVTDPERFGLTRDRWVAAAESSINHLASHPSTRPDVTFLATTNEEHQLGLTSQHLTCFDGGSFLLGGTLLDRQDFVDFGLDLVAGCHETTNSTLTGIGPEQFSWDPNGVPDSQKELFERAGFTINSGQTILRPEVIESFTTAWRVTGDGTTLEWVWNAFTNINKTCRTATGFAGLENVNAANGGGRIDNQESFMFAEVLKTSFLTFAPEDDWQVQKGSGNTFVTNTEAHPFKVTTPQ

SEQ ID NO: 7 - DNA, 6 bp, Aspergillus nidulans
gtaagt

SEQ ID NO: 8 - DNA, 6 bp, Aspergillus nidulans
  misc_feature (4)..(4): n is a, c, g, or t/u
gtangt

SEQ ID NO: 9 - DNA, 6 bp, Aspergillus nidulans
gctgac

SEQ ID NO: 10 - DNA, 6 bp, Aspergillus nidulans
  misc_feature (1)..(1): r is g or a
rctrac

SEQ ID NO: 11 - DNA, 6 bp, Aspergillus nidulans
gtacgt

SEQ ID NO: 12 - DNA, 6 bp, Aspergillus nidulans
actgac

SEQ ID NO: 13 - protein, 10 aa, Asperfillus nidulans
GGLGESFTET

SEQ ID NO: 14 - protein, 9 aa, Aspergillus nidulans
LAETLKTLT

SEQ ID NO: 15 - DNA, 29 bp, Artificial Sequence
  PCR Primer
ggyggyctnggygartcnttctacgagta

SEQ ID NO: 16 - DNA, 33 bp, Artificial Sequence
  PCR Primer
gtanaggtacttnagngtctcngcnagrhagaa

SEQ ID NO: 17 - DNA, 2032 bp, Aspergillus nidulans
  CDS (36)..(1793)
atcgatgtctgctgcataaaggcagacggaggaagatgccgagacggtggtcctccctcatcagcatcacagccatcttcttggtcctcttcttcctccttcataggaatacagacacaccacgcgccgccaatagggctacaaacggccctgccaacggctttgctaggcagcaaagcatatgtccatcaacaccccctcagcctccatataaccgaaccagcacgggagggttcaactggggtgaaatcccagtcagataccctgtatccgacttcatcccgctgtcaaccaactctcctgcaacacttccgcgcatccaacgctcttccttcccacttcaatcctcaatcactaaatcccgccaggcagcagtcaaaggtgcctttcagcgcgcatggacctcctacacaacccacgcctggaaggcggacgaggtacggcccatcacggccggatctcgaaacaactttggcggatggggagcgaccctagtcgacaatctcgacacactgctaatcatggggctggacgaggagttcgcagcggcagtcgacgcgctcgcagatatagaattcagcccgcactcgtccccatcctcctcccagagcacaatcaacatattcgaaacgacaatccggtatctgggcggcttgctcgcggcgtatgatctcactggctgtcgagagactcggctgctggacaaagcaatccagcttggggagatgatctacacctccttcgacacagagaaccgcatgcccgtaccacggtggaatctgcacaaagcaggcaacggagagcctcagcgcgcggcagtgcagggcgtgctcgctgaactcgccagcagcagtctcgagttcacgcggctgtcgcagctgacgggggatatgcggtatttcgatgcggcatcccgcattaccgatctgcttgactcccaagccggccatacccggatcccggggttgtggccagtcagcgtgaacctgcagaaaggcgatctgacccgtgggtcgacattcagttttggcgggatggccgatagcgcctacgagtatctcggcaagacgtatcggctcctcggtggtgtggggaaagggccacagtacgagcgtctggcgcgaaacgcactagatgccgggattcgacatctcctcttccgaccgatgacgcctgatcatgcagatatcctcctacccggggtcgcgcacgcaaccagctcttccgtgggactcgagccccggacagagcatctcgcctgttttgtgggtgggatgtacgcgctcgccgggaagcttttctcaaaccagacgtacctcgacaccggccggaagctgacagacggttgtatctggtactacgataattcaccgctaggtatcatgccggagatgttcaccgtgccggcttgtccgtcagtggctgaatgtccttgggacgaaacaaggggtggtatctacacctacgtgcgtgatgggcactactttctgcgtcctgaggcaatggagagtatcttctatatgtggcgcattacaggggacgaaaagtaccgcgaggctgcatggagaatgttcacggctatcgaagcggttacaaagacggagtttgggaatgcggcggtgcgggatgttatggttgaggaaggaaatgtaaagagagaagatagcatggagagtttctggatggcagagacgttgaagtatctgtatctgatatttggggagaccgatttggtcagcttggacgactgggtgttcaatacggaggcgcaccctttgaggggtgcagggagttgacattgtattcacacatcggtatagacaaattatagagtagacgttcaaaacggccaaaactgaatggatagactccatatgcattgaatatacaatgtattcgctgcaaagcatggataaaataaagatgtacaaagtgtctttgttgtcgctttgaaagtggtatatcatcccatcataaggtggcagtgtaaccaaccctctatatcacctacatagacagctgatagaccggc

SEQ ID NO: 18 - protein, 586 aa, Aspergillus nidulans
MPRRWSSLISITAIFLVLFFLLHRNTDTPRAANRATNGPANGFARQQSICPSTPPQPPYNRTSTGGFNWGEIPVRYPVSDFIPLSTNSPATLPRIQRSSFPLQSSITKSRQAAVKGAFQRAWTSYTTHAWKADEVRPITAGSRNNFGGWGATLVDNLDTLLIMGLDEEFAAAVDALADIEFSPHSSPSSSQSTINIFETTIRYLGGLLAAYDLTGCRETRLLDKAIQLGEMIYTSFDTENRMPVPRWNLHKAGNGEPQRAAVQGVLAELASSSLEFTRLSQLTGDMRYFDAASRITDLLDSQAGHTRIPGLWPVSVNLQKGDLTRGSTFSFGGMADSAYEYLGKTYRLLGGVGKGPQYERLARNALDAGIRHLLFRPMTPDHADILLPGVAHATSSSVGLEPRTEHLACFVGGMYALAGKLFSNQTYLDTGRKLTDGCIWYYDNSPLGIMPEMFTVPACPSVAECPWDETRGGIYTYVRDGHYFLRPEAMESIFYMWRITGDEKYREAAWRMFTAIEAVTKTEFGNAAVRDVMVEEGNVKREDSMESFWMAETLKYLYLIFGETDLVSLDDWVFNTEAHPLRGAGS

SEQ ID NO: 19 - DNA, 6 bp, Artificial Sequence
  Consensus Splice Site
rctrac

SEQ ID NO: 20 - protein, 18 aa, Artificial
  synthetic signal sequence
MDRFLGRHLGLLRHCLRQ

SEQ ID NO: 21 - protein, 18 aa, Artificial
  synthetic signal sequence
MDRFLAVISAFFATAFAK

SEQ ID NO: 22 - DNA, 81 bp, Artificial
  primer
gggaaaccatggaccggttcctcgccgtcatctcggccttcttcgccactgccttcgccaagcgcccggtgatgcgcagtg

SEQ ID NO: 23 - DNA, 21 bp, Artificial
  primer
cacctcggcgaggttcttgcg

SEQ ID NO: 24 - DNA, 83 bp, Artificial
  primer
gggaaaccatggaccggttcctcgccgtcatctcggccttcttcgccactgccttcgccaagacaccacgcgccgccaatagc

SEQ ID NO: 25 - DNA, 21 bp, Artificial
  primer
ggaggatggggacgagtgcgg

(19) United States
(12) Patent Application Publication
(10) Pub. No.: US 2005/0260627 A1
(43) Pub. Date: Nov. 24, 2005

(54) Mannosidases and methods for using same

(75) Inventors: William E. Hintz, Victoria (CA); Caleb Joshua Eades, Victoria (CA)

Correspondence Address: KLARQUIST SPARKMAN, LLP, 121 SW SALMON STREET, SUITE 1600, PORTLAND, OR, 97204, US

(73) Assignee: University of Victoria Innovation and Development Corporation

(21) Appl. No.: 11/073,112

(22) Filed: Mar. 3, 2005

Related U.S. Application Data
(63) Continuation-in-part of application No. 10/089,211, filed on Mar. 25, 2002, now abandoned.
(60) Provisional application No. 60/157,341, filed on Oct. 1, 1999.

(51) Int. Cl. C12Q 1/68; C12P 19/18; C12P 19/34

(52) U.S. Cl. 435/6; 435/91.2

(57) ABSTRACT

Mannosidase enzymes and use of such enzymes to alter the glycosylation patterns of macromolecules are disclosed. Also disclosed are the nucleic acid sequences encoding the mannosidase enzymes.

### CROSS REFERENCE TO RELATED APPLICATIONS

This application is a continuation in part of U.S. application Ser. No. 10/089,211, filed 25 Mar. 2002, which is the US National Stage of International Application No. PCT/US00/27210, filed Oct. 2, 2000, which in turn claims the benefit of U.S. Provisional Application No. 60/157,341, filed Oct. 1, 1999, the disclosure of which is incorporated herein by reference.

### FIELD

The application relates to mannosidases and methods of using mannosidases for altering the glycosylation pattern of macromolecules.

### BACKGROUND

The N-linked protein glycosylation pathways are fairly well characterized in higher eukaryotes (Kornfeld and Kornfeld, Ann. Rev. Biochem. 54: 631-664,1985), however, less is known about such pathways in lower eukaryotes. What is known about protein glycosylation in lower eukaryotes has come largely from studies of the yeast Saccharomyces cerevisiae and may not be sufficient to describe N-linked glycosylation in other lower eukaryotes, such as filamentous fungi.

Evolutionary studies suggest that the filamentous ascomycetes diverged from yeasts from 400 million years ago (Berbee and Taylor, Mol. Biol. Evol. 9: 278-284, 1992) to 1 billion years ago, the latter being about the time the fungal branch split from plants and animals (Doolittle et al., Science 271: 470-477,1996). Filamentous fungi produce N-glycan structures that appear to be different than those produced in yeast, suggesting a different mode of formation of these structures than in yeast.

Studies have shown the presence in filamentous fungi of N-glycan structures containing five mannose units(Man5GlcNAc2), suggesting processing of a Man5GlcNAc2 precursor (Mares et al., Eur. J. Biochem. 245: 617-625,1997; Chiba et al., Curr. Microbiol. 27: 281-288,1993). The mechanisms of synthesis of high mannose N-glycans in filamentous fungi seem to differ from corresponding mechanisms in yeast, and may be more similar to processes in higher eukaryotes. Full characterization of the N-glycosylation pathways in these organisms is very important, since protein glycosylation plays an integral role in processes such as pathogenicity and protein secretion.

The early steps of the asparagine-linked (N-linked) protein glycosylation pathways are similar in higher and lower eukaryotes (see reviews in Kornfeld and Kornfeld, Ann. Rev. Biochem. 54: 631-664,1985; Moreman et al., Glycobiol. 4: 113 125,1994; Herscovics and Orlean, FASEB J. 7: 540-550,1993; Herscovics, Biochim. Biophys. Acta 1426: 275-285, 1999). Initially, an oligosaccharide precursor consisting of three glucose, nine mannose, and two N-acetylglucosamine molecules (Glc3Man9GlcNAc2) is co-translationally transferred to the newly synthesized polypeptide in the endoplasmic reticulum (ER). In the ER,α-glucosidases I and II first remove the three glucose molecules. In the ER and/or Golgi apparatus, α-mannosidase (s) then remove one or more of the mannose residues from the precursor. In higher eukaryotes (i.e., mammals), α-1,2-mannosidases remove a total of four mannose residues, yielding Man5GlcNAc2 which is the precursor for complex, hybrid, and high-mannoseN-glycans. In the yeast S. cerevisiae, however, an ER-specific mannosidase, α-1,2-mannosidase, removes only a single mannose residue, producing Man8GlcNAc2 (Herscovics, Biochim. Biophys. Acta 1426: 275285,1999).

Subsequent steps in the pathways in higher and lower eukaryotes are quite different. In higher eukaryotes, following the addition of a single GlcNAc to Man5GlcNAc2 by GlcNAc transferase I (GnTI), mannosidase II removes two additional mannose groups, producing GlcNAcMan3GlcNAc2. Various transferases, such as GnTII, fucosyl transferase, galactosyl transferase, and sialyl transferase, assemble the oligosaccharide into its final structure. In higher eukaryotes a variety of different carbohydrate units can thus be attached to a common precursor to form an array of distinct N-glycans. In S. cerevisiae, after the removal of a single mannose, various mannosyltransferases then add additional mannose units to Man8GlcNAc2 to form large high-mannose N-glycans containing up to 13 mannose units, and even larger mannan outer chains containing up to 200 mannose residues (Herscovics and Orlean, FASEB J. 7: 540-550,1993). The removal of mannose residues from the glycan chain during the initial stages of processing appears to be significantly different for higher and lower eukaryotes, resulting in quite different N-glycan structures for these organisms. Based on the evolutionary history of filamentous fungi, their N-glycan processing is likely to contain elements of the pathways in both mammals and yeast. Filamentous fungi do not produce complex N-glycans because such fungi lack the further processive transferases; however, the initial oligosaccharide precursor is trimmed to Man5GlcNAc2.

The α-mannosidases have been classified previously into two independently derived groups, Class 1 and Class 2, based on biochemical properties, substrate specificity, inhibitor profiles, and sequence alignments, (Daniel et al., Glycobiol. 4: 551-566,1994; Moreman et al., Glycobiol. 4: 113-125,1994; Eades et al., Glycobiol. 8: 17-33,1998). The first group contains the α-1,2-mannosidases found in the ER and the Golgi apparatus, including the ER Man9-mannosidase, ER endomannosidase, and the Golgi mannosidasel. The second group of a mannosidases is more heterogeneous and contains the lysosomal mannosidases, the Golgi mannosidaseII, and a distantly related group of enzymes, including the rat ER/cytosolic mannosidase (Bischoffet al., J. Biological Chem. 28: 17110-17117, 1990), yeast vacuolar mannosidase (Yoshihisa and Anraku, Biochem. Biophys. Res. Comm. 163: 908-915,1989), and the A. nidulans Class 2 mannosidase (Eades et al., Glycobiol. 8: 17-33,1998).

### SUMMARY

This application stems from the discovery of the amino acid sequences and corresponding nucleic acid sequences for all members of a mannosidase gene family of Aspergillus nidulans. These mannosidases are particularly useful for altering the glycosylation patterns of macromolecules such as proteins. Glycosylation affects many properties of a glycoprotein, including protein folding, protease resistance, intercellular trafficking, compartmentalization, secretion, inter-and intra-molecular associations, intermolecular affinities, tissue targeting and biological half-life. Glycosylation patterns may also significantly alter the biological activity, solubility, clearance, intermolecular aggregation, and antigenicity, especially for those proteins having therapeutic utility. Thus, the present disclosure enables glycoproteins to be engineered to be more effectively used and produced.

One aspect of the present disclosure provides the amino acid sequences of a novel α-1,2-mannosidase protein, which would not be predicted from the prior art. The disclosure also provides variants of the disclosed mannosidase protein. These variants can differ from the disclosed sequences by one or more conservative amino acid substitutions. Additionally, the disclosure provides variants of the disclosed mannosidase protein having at least 80% sequence identity to the disclosed mannosidase amino acid sequences such as at least 90% or at least 95% sequence identity.

According to another aspect of the disclosure, respective nucleic acid sequences are provided that encodes the α-1,2-mannosidase summarized above. These nucleic acid sequences can be operably linked to control sequences and incorporated into any of various vectors. The resulting recombinant vectors are useful for transforming any of various host cells. Once transformed, the host cell can produce the α-1,2-mannosidase of the present disclosure. Host cells can be obtained from fungi, plants, bacteria, animals, algae, yeast, and insects.

According to yet another aspect of the disclosure, methods are provided for altering the glycosylation pattern of target proteins using the α-1,2-mannosidase of the present disclosure. The α-1,2-mannosidase can be placed in contact with a protein and allowed to alter the glycosylation pattern of the protein. The methods can be practiced in vivo by creating a transgenic host cell that overexpresses or under-expresses the α-1,2-mannosidase of the present disclosure.

According to yet another aspect of the disclosure, isolated nucleic acid molecules and amino acid molecules are provided that have at least 80% sequence identity to in SEQ ID NO 17; or the complementary strands thereof such as at least 90% or at least 95% sequence identity.

### BRIEF DESCRIPTION OF THE DRAWINGS

FIGS. 1A-1J show a DNA sequence of A. nidulans mannosidase 1A.

The derived amino acid sequence is indicated by single-letter designations. The hydrophobic transmembrane region is indicated by a dotted underline. Upstream elements are underlined. The intron is indicated in bold type and consensus splice sites are underlined. The stop codon is indicated by a triple asterisk(***).

FIGS. 2A-2G show a DNA sequence of A. nidulans mannosidase 1B. The derived amino acid sequence is indicated by single-letter designations. Upstream elements are underlined. The introns are indicated in bold type and consensus splice sites are underlined. The stop codon is indicated by a triple asterisk (***).

FIG. 3 shows a Kyte-Doolittle hydropathy plot of the predicted amino acid sequences of (A) A. nidulans mannosidase 1A and (B) A. nidulans mannosidase 1B. The vertical axis is the hydrophobicity of a given region of the protein (Kyte and Doolittle, J. Mol. Biol. 157: 105-132,1982) with positive values representing hydrophobic regions of the protein, and negative values representing hydrophilic regions of the protein.

FIG. 4 shows a sequence-similarity matrix generated from the ClustalW (Thompson et al., Nucl. Acids Research 22: 4673-4680,1994) sequence alignment of 13 Class 1 α-mannosidases and the pairwise percent similarity of the amino acid sequences for all possible pairs of sequences.

FIG. 5 shows a dendrogram showing sequence relationships of mannosidases. The dendrogram was generated from the ClustalW sequence alignment. Three major groups of related enzymes are shown in shaded boxes.

FIGS. 6A and 6B show the amino acid and nucleic acid sequences of mannosidase 1C.

FIG. 7: Construction of secretion vectors for the Aspergillus nidulans α-1,2-mannosidase IB and α-1,2-mannosidase 1C enzymes. The first 73 bp of the α-1,2-mannosidase IB coding sequence which encodes the transmembrane region of the protein (TM) was replaced by a 54 bp synthetic signal sequence (SS) which was ligated to 2002 base pairs of the inducible alcA promoter. Similarly, the first 91 bp of the α-1,2-mannosidase IC coding sequence was replaced with the 54 bp synthetic signal sequence and ligated to the alcA promoter. The base positions of the new coding regions in the secretion vectors is shown, while the base positions from original gene sequence are shown in brackets.

FIG. 8: Biochemical characterization of purified A. nidulans α-1,2-mannosidase IB (A) and α-1,2-mannosidase IC (B) enzymes. Enzyme activity was assayed by digestion of the synthetic substrate Man-α1,2-Man-OCH3 at 37oC for 3 hours. Released mannose was detected using a colorimetric glucose oxidase/horseradish peroxidase assay (see Materials and Methods) and measuring absorbance at 405 nm.

FIG. 9: Partial digestion of Man9GlcNAc2 (M9) with A. nidulans α-1,2-mannosidase IB (A) and A. nidulans α-1,2-mannosidase IC (B) by MALDI-TOF mass spectrometry. Peaks represent parent molecules ionized with sodium (M+Na+). Several different digestion products are produced in each reaction, including Man8GlcNAc2 (M8), Man7GlcNAc2 (M7), Man6GlcNAc2 (M6) and Man5GlcNAc2 (M5).

### SEQUENCE LISTING

The nucleic and amino acid sequences listed in the accompanying sequence listing are shown using standard letter abbreviations for nucleotide bases, and three letter code for amino acids. Only one strand of each nucleic acid sequence is shown, but the complementary strand is understood as included by any reference to the displayed strand.

SEQ ID NO:1 is the nucleic acid sequence of the mannosidase 1A gene.

SEQ ID NO: 2 is the nucleic acid sequence of the mannosidase 1A cDNA.

SEQ ID NO: 3 is the deduced amino acid sequence of mannosidase 1A.

SEQ ID NO: 4 is the nucleic acid sequence of the mannosidase 1B gene.

SEQ ID NO: 5 is the nucleic acid sequence of the mannosidase 1B cDNA.

SEQ ID NO: 6 is the deduced amino acid sequence of mannosidase 1B.

SEQ ID NO: 7 is a 5′-splice site sequence of the mannosidase 1A gene.

SEQ ID NO: 8 is a 5′-splice site consensus sequence of filamentous fungi.

SEQ ID NO: 9 is an internal lariat sequence of the mannosidase 1A gene.

SEQ ID NO: 10 is an internal lariat consensus sequence of filamentous fungi.

SEQ ID NO: 11 is a 5′-splice site sequence of the mannosidase 1B gene.

SEQ ID NO: 12 is an internal lariat sequence of the mannosidase 1B gene.

SEQ ID NO: 13 is the amino acid sequence used to generate a forward primer.

SEQ ID NO: 14 is the amino acid sequence used to generate a reverse primer.

SEQ ID NO: 15 is a PCR primer useful for identifying mannosidases.

SEQ ID NO: 16 is a PCR primer useful for identifying mannosidases.

SEQ ID NO: 17 is the nucleic acid sequence of the mannosidase 1C gene.

SEQ ID NO: 18 is the deduced amino acid sequence of mannosidase 1C.

SEQ ID NO: 19 is the deduced amino acid sequence of a consensus splice site.

SEQ ID NOS: 20 and 21 are secretion signal sequences.

SEQ ID NOS: 22-25 are primer sequences.

### DETAILED DESCRIPTION

I. Definitions

“Sequence Identity.” The similarity between two nucleic acid sequences or between two amino acid sequences is expressed in terms of the level of sequence identity shared between the sequences. Sequence identity is typically expressed in terms of percentage identity; the higher the percentage, the more similar the two sequences are. Methods for aligning sequences for comparison purposes are well known in the art. Various programs and alignment algorithms are described in: Smith & Waterman, Adv. Appl. Math. 2: 482,; Needleman & Wunsch, J. Mol. Biol. 48: 443,1970; Pearson & Lipman, Proc. Natl. Acad. Sci. USA 85: 2444,1988; Higgins & Sharp, Gene 73: 237-244,1988; Higgins & Sharp, CABIOS 5: 151-153, 1989; Corpet et al., Nucleic Acids Research 16: 10881-10890,1988; Huang, et al., Computer Applications in the Biosciences 8: 155-165,1992; and Pearson et al., Methods in Molecular Biology 24: 307-331,1994. Altschul et al., J. Mol. Biol., 215: 403-410,1990, presents a detailed consideration of sequence alignment methods and homology calculations. The NCBI Basic Local Alignment Search Tool (BLAST, Altschul et al. J. Mol. Biol., 215: 403-410,1990) is available from several sources, including the National Center for Biotechnology Information (NCBI, Bethesda, Md.) and on the Internet, for use in connection with the sequence-analysis programs blastp, blastn, blastx, tblastn and tblastx.BLASTTM can be accessed on the NCBI website. A description of how to determine sequence identity using this program is available on the internet at the NCBI website.

For comparisons of amino acid sequences of greater than about 30 amino acids, the “Blast 2 sequences” function in the BLAST program is employed using the default BLOSUM62 matrix set to default parameters, (gap existence cost of 11, and a per-residue gap cost of 1). When aligning short peptides (fewer than about 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 45%, at least 50%, at least 60%, at least 70%, at least 75%, at least 80%, at least 85%, at least 90%, or at least 95% sequence identity.

“Substantial similarity” A first nucleic acid is “substantially similar” to a second nucleic acid if, when optimally aligned (with appropriate nucleotide insertions or deletions) with the other nucleic acid (or its complementary strand), there is nucleotide sequence identity in at least about 60%, 75%, 80%, 85%, 90% or 95% of the nucleotide bases. Sequence similarity can be determined by comparing the nucleotide sequences of two nucleic acids using the BLAST sequence analysis software (blastn) available from The National Center for Biotechnology Information. Such comparisons may be made using the software set to default settings (expect=10, filter=default, descriptions=500 pairwise, alignments=500, alignment view=standard, gap existence cost=11, per residue existence=1, per residue gap cost=0.85). Similarly, a first polypeptide is substantially similar to a second polypeptide if it shows sequence identity of at least about 75%-90% or greater when optically aligned and compared using BLAST software (blastp) using default settings.

“Specific Binding Agent.” A “specific binding agent” is an agent that is capable of specifically binding to the mannosidases of the present disclosure, and may include polyclonal antibodies, monoclonal antibodies (including humanized monoclonal antibodies) and fragments of monoclonal antibodies such as Fab, F (ab′) 2 and Fv fragments, as well as any other agent capable of specifically binding to the epitopes on the proteins.

“Operably linked.” A first nucleic acid sequence is “operably linked” with a second nucleic acid sequence whenever the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

“Isolated.” An “isolated” biological component (such as a nucleic acid or protein or organelle) is a component that has been substantially separated or purified away from other biological components in the cell of the organism in which the component naturally occurs, i. e., other chromosomal and extra-chromosomal DNA,

RNA, proteins, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids.

“Recombinant.” A “recombinant” nucleic acid is one having a sequence that is not naturally occurring or has a sequence made by an artificial combination of two otherwise-separated, shorter sequences. This artificial combination is often accomplished by chemical synthesis or, more commonly, by the artificial manipulation of isolated segments of nucleic acids, e. g., by genetic engineering techniques.

“Transformed.” A “transformed” cell is a cell into which a nucleic acid molecule has been introduced by molecular biology techniques. As used herein, the term “transformation” encompasses all techniques by which a nucleic acid molecule can be introduced into such a cell, including transfection with a viral vector, transformation with a plasmid vector, and introduction of naked DNA by electroporation, lipofection, and particle gun acceleration.

“Vector.” A “vector” is a nucleic acid molecule as introduced into a host cell, thereby producing a transformed host cell. A vector may include nucleic acid sequences, such as an origin of replication, that permit the vector to replicate in a host cell. A vector may also include one or more selectable marker genes and other genetic elements known in the art.

“Glycosylation pattern.” The “glycosylation pattern” is the characteristic structure (including branch structure), number, or location of oligosaccharide structures associated with a macromolecule, such as a protein.

“DNA construct.” The term “DNA construct” is intended to denote any nucleic acid molecule of cDNA, genomic DNA, synthetic DNA or RNA origin. The term “construct” is intended to denote a nucleic acid segment that may be single-or double-stranded, and that may be based on a complete or partial naturally occurring nucleotide sequence encoding one or more of the mannosidase genes of the present disclosure. It is understood that such nucleotide sequences include intentionally manipulated nucleotide sequences, e. g., subjected to site-directed mutagenesis, and sequences that are degenerate as a result of the genetic code. All degenerate nucleotide sequences are included within the scope of the disclosure so long as the mannosidase enzyme encoded by the nucleotide sequence maintains the ability to hydrolytically remove terminal mannoside residues.

“Mannosidase activity.” The phrase “mannosidase activity” describes the enzymatic catalysis of the hydrolytic removal of terminal mannoside residues. “Probes and primers. “Nucleic acid probes and primers may be readily prepared based on the nucleic acid sequences provided by this disclosure. A “probe” comprises an isolated nucleic acid sequence attached to a detectable label or reporter molecule. Typical labels include radioactive isotopes, ligands, chemiluminescent agents, and enzymes. Methods for labeling and guidance in the choice of labels appropriate for various purposes are discussed, e. g., in Sambrook et al. (eds.),

Molecular Cloning : A Laboratory Manual, 2nd ed., vol. 1-3, Cold Spring Harbor Laboratory Press, Cold Spring Harbor, N.Y., 1989; and Ausubel et al. (ed.) Current Protocols in Molecular Biology, Greene Publishing and Wiley-Interscience, New York (with periodic updates), 1987.

“Primers” are short nucleic acids, preferably DNA oligonucleotides 15 nucleotides or more in length, that are annealed to a complementary target DNA strand by nucleic acid hybridization to form a hybrid between the primer and the target DNA strand, then extended along the target DNA strand by a DNA polymerase enzyme. Primer pairs can be used for amplification of a nucleic acid sequence, e. g., by the polymerase chain reaction (PCR) or other nucleic-acid amplification methods known in the art.

As noted, probes and primers are preferably 15 nucleotides or more in length, but, to enhance specificity, probes and primers of 20 or more nucleotides may be preferred.

Methods for preparing and using probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989; Ausubel et al. (ed.), Current Protocols in Molecular Biology, Greene Publishing and Wiley-Interscience, N.Y. (with periodic updates), 1987; and Innis et al., PCR Protocols : A Guide to Methods and Applications, Academic Press: San Diego, 1990. PCR primer pairs can be derived from a known sequence, for example, by using computer programs intended for that purpose such as Primer (Version 0.5, 1991, Whitehead Institute for Biomedical Research, Cambridge, Mass.). One of skill in the art will appreciate that the specificity of a particular probe or primer increases with the length of the probe or primer. For example, a primer comprising 20 consecutive nucleotides will anneal to a target with a higher specificity than a corresponding primer of only 15 nucleotides. Thus, in order to obtain greater specificity, probes and primers may be selected that comprise, by way of example, 10, 20, 25, 30, 35, 40, 50 or more consecutive nucleotides.

II. Methods of Producing Mannosidase 1A, 1B, and 1C

A. Cloning Nucleic Acid Sequences Encoding Mannosidase

Provided with the nucleic acid sequences of the genes encoding the mannosidases 1A, 1B, and 1C (SEQ ID NOS: 1,4, and 17, respectively), one of ordinary skill in the art will appreciate that several different methods can be used to isolate the genes and the cDNAs encoding the corresponding mannosidases. One example of such a method is the polymerase chain reaction (PCR) (U. S. Pat. No. 4,683,202 to Mullis; and Saiki et al., Science 239: 487-491,1988).

When using PCR to isolate a sequence encoding the gene, a primer can be designed that targets the extreme 5′end of the sequence, and a second primer can be designed that targets the extreme 3′end of the sequence. For example the 5′primer(5′-GGYGGYCTNGGYGARTCNTTCTACGAGTA-3′; SEQ ID NO: 15) and the 3′primer(5′-GTANAGGTACTTNAGNGTCTCNGCNAGRHAGAA-3′; SEQ ID NO: 16) are used in a PCR (polymerase chain reaction) procedure to generate multiple copies of the gene. The copies are isolated by separation on an agarose gel. The fragment of interest is then removed from the gel, and ligated into an appropriate vector. Alternatively, the gene can be created by engineering synthetic strands of DNA that partially overlap each other (Beaucage & Caruthers, Tetrahedron Letters 22: 1859-1869,1981; Matthes et al., Embo. J. 3: 801-805,1984). The synthetic strands are annealed and a DNA polymerase is used to fill in the single-stranded regions. The resulting synthetic double-stranded DNA molecule can be cloned into a vector.

For use as primers and probes, nucleic acid sequences can contain at least 15 contiguous nucleic acid molecules of either of the sequences shown in Seq. ID. No. 1 and SEQ ID NO: 4, or either of the complementary strands of the molecules shown in SEQ ID NO: 1 and SEQ ID NO: 4. The nucleic acid sequences are useful for performing hybridization protocols, such as Northern blots or Southern blots as described in Sambrook et al., (eds.), Molecular Cloning, A Laboratory Manual, 2d ed., vol. 1-3, Cold Spring Harbor Laboratory Press, Cold Spring Harbor, N.Y., 1989. These hybridization protocols can be used to identify nucleic acid sequences that are substantially similar to those shown in SEQ ID NOS: 1, 4, or 17. A successful hybridization to such sequences indicates that the analogous nucleic acid sequence hybridizes to the oligonucleotide probe that comprises at least a fragment of the sequences shown in SEQ ID NOS: 1, 4, or 17. Generally hybridization conditions are classified into categories, for example very high stringency, high stringency, and low stringency. The conditions corresponding to these categories are provided below.

Very High Stringency (detects sequences that share 90% sequence identity). Hybridization in 5×SSC at 65 C 16 hours. Wash twice in 2×SSC at room temp. 15 minutes each. Wash twice in 0.2×SSC at 65 C 20 minutes each.

High Stringency (detects sequences that share 80% sequence identity or greater). Hybridization in 3×SSC at 65 C 16 hours. Wash twice in 2×SSC at room temp. 15 minutes each. Wash twice in 0.5×SSC at 55 C 20 minutes each

Low Stringency (detects sequences that share greater than 50% sequence identity). Hybridization in 3×SSC at 65 C 16 hours. Wash twice in 2×SSC at room temp. 20 minutes each.

Mannosidase-encoding nucleic acid sequences according to the disclosure also encompass mannosidase enzymes that differ in amino acid sequence from the mannosidase 1A, 1B, and 1C sequences of SEQ ID NOS: 1, 4, and 17, and that maintain mannosidase activity. Such proteins may be produced by changing the cDNA nucleotide sequence of mannosidase 1A, 1B, or 1C, by changing the sequence of the respective genes using standard procedures such as site-directed mutagenesis, or by performing the polymerase chain reaction. The simplest modifications involve substituting one or more amino acids with other amino acids having similar biochemical properties. These so-called “conservative substitutions” usually have minimal impact on the activity of the resultant protein. Table 1 shows amino acids that may be substituted for an original amino acid in a protein and that are regarded as conservative substitutions.

TABLE 1

| Original | Conservative |
|---|---|
| Residue | Substitutions |
| ala | ser |
| arg | lys |
| asn | gln; his |
| asp | glu |
| cys | ser |
| gln | asn |
| glu | asp |
| gly | pro |
| his | asn; gln |
| ile | leu; val |
| leu | ile; val |
| lys | arg; gln; glu |
| met | leu; ile |
| phe | met; leu; tyr |
| ser | thr |
| thr | ser |
| trp | tyr |
| tyr | trp; phe |
| val | ile; leu |

More substantial changes in enzymatic function or other features may be obtained by selecting substitutions that are less conservative than those in Table 1, that is, selecting residues that differ more significantly in their effect on maintaining: (a) the structure of the polypeptide backbone in the area of the substitution, for example, as a sheet or helical co generally produce the greatest changes in protein properties are those in which: (a) a hydrophilic residue, such as, seryl or threonyl, is substituted for (or by) a hydrophobic residue, such as, leucyl, isoleucyl, phenylalanyl, valyl, or alanyl; (b) a cysteine or proline is substituted for (or by) any other residue; (c) a residue having an electropositive side chain, such as, lysyl, arginyl, or histadyl, is substituted for (or by) an electronegative residue, such as, glutamyl or aspartyl; or (d) a residue having a bulky side chain, such as, phenylalanine, is substituted for (or by) one not having a side chain, such as, glycine. The effects of these amino acid substitutions or deletions or additions may be assessed for mannosidase protein derivatives by analyzing the ability of the respective modified polypeptide to catalyze the removal of terminal mannoside residues.

Variant mannosidase cDNA or genes may be produced by standard DNA mutagenesis techniques, for example, M 13 primer mutagenesis. Details of these techniques are provided in Sambrook et al. (ed.), Molecular Cloning : A Laboratory Manual, 2nd ed., vol. 1-3, Ch. 15, Cold Spring Harbor Laboratory Press, Cold Spring Harbor, N.Y., 1989, and Ausubel et al. (ed.) Current Protocols in Molecular Biology, Greene Publishing and Wiley-Interscience, N.Y. (with periodic updates), 1987. By the use of such techniques, variants may be created that differ slightly from the mannosidase cDNA or gene sequences specifically disclosed, yet that still encode a protein having mannosidase activity. DNA molecules and nucleotide sequences that are derivatives of those specifically disclosed herein and that differ from those disclosed by the deletion, addition, or substitution of nucleotides while still encoding a protein having mannosidase activity are comprehended by this disclosure. In their simplest form, such variants may differ from the disclosed sequences by alteration of the coding region to fit the codon usage bias of the particular organism into which the molecule is to be introduced. Alternatively, the coding region may be altered by taking advantage of the degeneracy of the genetic code to alter the coding sequence in such a way that, while the nucleotide sequence is substantially altered, it nevertheless encodes a protein having an amino acid sequence identical or substantially similar to the mannosidase 1A and 1B sequences specifically disclosed in SEQ ID NOS: 3 and 6. For example, the fourth amino acid residue of the mannosidaseIA gene is alanine and is encoded in the mannosidasel A open reading frame (ORF) by the nucleotide codon triplet GCA. Because of the degeneracy of the genetic code, three other nucleotide codon triplets-GCT, GCC and GCG-also code for alanine. Thus, the nucleotide sequence of the mannosidase 1A ORF could be changed at this position to any of these three codons without affecting the amino acid composition of the encoded protein or the characteristics of the protein. Based upon the degeneracy of the genetic code, variant DNA molecules may be derived from the cDNA and gene sequences disclosed herein using standard DNA mutagenesis techniques as described above, or by synthesis of modified DNA sequences. Thus, this disclosure also encompasses nucleic acid sequences that encode either a mannosidase 1A or 1B protein but that vary from the disclosed nucleic acid sequences due to the degeneracy of the genetic code.

B. Vectors

The choice of expression vector depends in part on the type of host cell that will be used for the expression of the mannosidase enzyme. The promoter sequence used must be recognizable by the enzymes responsible for translation within the host cell. One of skill in the art will appreciate that there are a number of regulatory sequences known in the art that function in conjunction with bacterial host cells, plant host cells, insect host cells, yeast host cells, and fungal host cells. Examples of constitutive plant promoters that may be useful for expressing the cDNA include: the cauliflower mosaic virus (CaMV) 35S promoter, which confers constitutive, high-level expression in most plant tissues (see, e. g., Odel et al., Nature 313: 810,1985), nopaline synthase promoter (An et al., Plant Physiol. 88: 547,1988); and the octopine synthase promoter (Fromm et al., Plant Cell 1: 977, 1989). Examples of suitable promoters for use in filamentous fungus host cells are, for instance, the ADH3 promoter (McKnight et al., Embo. J. 4: 2093-2099,1985) or the tpiA promoter (Blattner et al., Science 27: 1453-1474,1997). Examples of other useful promoters are those derived from the genes encoding A. oryzae TAKA amylase, Rhizomucor miehei aspartic proteinase, A. niger neutrala-amylase, A. niger acid-stable a-amylase, A. niger or A. awamoriglucoamylase (gluA), Rhizomucor miehei lipase, A. oryzae alkaline protease, A. oryzae triose phosphate isomerase, or A. nidulans acetamidase. It is also possible that the native mannosidase promoter could be used to express the enzyme. Similarly, one of ordinary skill in the art will appreciate that there are several promoters available that can be used in insect expression systems (such as, baculovirus) and bacterial expression systems.

To direct the enzyme into the secretory pathway of the host cell, a secretory signal sequence (also known as a leader sequence, “prepro” sequence or “pre” sequence) can be provided in the vector. The secretory signal sequence is joined to the DNA sequence encoding the enzyme in the correct reading frame. Secretory signal sequences are commonly positioned 5′ to the DNA sequence encoding the enzyme. The secretory signal sequence may be the sequences normally associated with the enzyme or from a gene encoding another secreted protein.

The vector can also contain a selectable marker. The selectable marker allows host cells that have been successfully transformed with the construct of interest to be identified. Selectable markers for use in plant cells are, for example Bostar, Kanr, and various other herbicide resistance genes. Selectable markers for use in mammalian cells are, for example, Ampr and Kanr. Selectable markers for use in filamentous fungi include, for example, amdS, pyrg, argB, niaD and sC.

C. Transformation

The DNA construct of the disclosure may be either homologous or heterologous to the host in question. If homologous to the host cell (produced by the host cell in nature), the construct typically will be operably connected to another promoter sequence or, if applicable, another secretory signal sequence and/or terminator sequence than in its natural environment. In this context, the term “homologous” is intended to include a cDNA sequence encoding the mannosidase 1A, 1B, or 1C native to the host A. nidulans. The term “heterologous” is intended to include a DNA sequence not expressed by the host cell in nature. Thus, the DNA sequence may be from another organism, or it may be a synthetic sequence. The host cell of the disclosure, into which the DNA construct or the recombinant expression vector of the disclosure is to be introduced, may be any cell capable of producing the disclosed mannosidase enzymes. Such cells include bacteria cells, yeast cells, fungal cells, insect cells, and higher eukaryotic cells.

Various methods of introducing the DNA construct into host cells are well known in the art. For example, in some species, the Ti plasmid of A. tumefaciens can be used to transform host cells (Gouka et al., Nature Biotechnology 6: 598-602, 1999), the host cell can also be transformed using gene blasting techniques and standard chemical treatments.

D. Production

The production of recombinant mannosidase 1A, 1B, or 1C may be accomplished using any of various transformed hosts, such as bacteria, plants, mammalian cell culture, whole mammals, insects, yeast, and fungi. As mentioned above, the selection of suitable promoter sequences and other regulatory elements will be specific to the host organism that will be used to produce the protein.

Additionally, within each group of potential hosts there are several species that can potentially be used to produce the protein. For example, filamentous fungi, such as, Aspergillus spp., Neurospora spp., Fusarium spp. or Trichoderma spp., can be used to produce mannosidase. More specifically, individual strains of Aspergillus, such as A. oryzae, A. nidulans, or A. niger, can be used. The use of Aspergillus spp. For the expression of proteins is described in, for example, EP 272,277 and EP 230,023. Transformation techniques specific for the transformation of F. oxysporum have been described by Malardier et al., Gene 78: 147-156,1988. Various yeast strains and yeast-derived vectors are commonly used for the expression of heterologous proteins. For instance, Pichia pastoris expression systems, obtained from Invitrogen (San Diego, Calif.), may be used to practice the present disclosure. Such systems include suitable Pichia pastoris strains, vectors, reagents, transformants, sequencing primers, and media. Available strains include KM71H a prototrophic strain, SMD1168H a prototrophic strain, and SMD1168 a pep4 mutant strain (Invitrogen Product Catalogue, 1998, Invitrogen, Carlsbad Calif.).

Non-yeast eukaryotic vectors may be used with equal facility for expression of proteins encoded by modified nucleotides according to the disclosure. Mammalian vector/host cell systems containing genetic and cellular control elements capable of carrying out transcription, translation, and post-translational modification are well known in the art. Examples of such systems are the well known Baculovirus system, the ecdysone-inducible mammalian expression system that uses regulatory elements from Drosophila melanogaster to allow control of gene expression, and the Sindbis viral-expression system that allows high-level expression in a variety of mammalian cell lines, all of which are available from Invitrogen.

The cloned expression vector can be transformed into any of various cell types for expression of the cloned nucleotide. Many different types of cells may be used to express modified nucleic acid molecules. Examples include cells of yeasts, fungi, insects, mammals, and plants, including transformed and non-transformed cells. For instance, common mammalian cells that could be used for the disclosure include HeLa cells, SW-527 cells (ATCC deposit#7940), WISH cells (ATCC deposit#CCL-25), Daudi cells (ATCC deposit#CCL-213), Mandin-Darby bovine kidney cells (ATCC deposit#CCL-22), and Chinese hamster ovary (CHO) cells (ATCC deposit#CRL-2092). Common yeast cells include Pichia pastoris (ATCC deposit#201178) and Saccharomyces cerevisiae (ATCC deposit#46024). Insect cells include cells from Drosophila melanogaster (ATCC deposit#CRL-10191), the cottonbollworm (ATCC deposit#CRL-9281) and from Trichoplusia ni egg cell homoflagellates. Fish cells that may be used include those from rainbow trout (ATCC deposit <RTI #CLL-55), salmon (ATCC deposit#CRL-1681), and zebrafish (ATCC deposit#CRL-2147). Amphibian cells that may be used include those of the bullfrog, Rana catesbelana (ATCC deposit#CLL-41). Reptile cells that may be used include those from Russell's viper (ATCC deposit#CCL-140). Plant cells that could be used include Chlamydomonas cells (ATCC deposit#30485), Arabidopsis cells (ATCC deposit#54069) and tomato plant cells (ATCC deposit#54003). Many of these cell types are commonly used and are available from the ATCC as well as from commercial suppliers such as Pharmacia (Uppsala, Sweden), and Invitrogen (San Diego, Calif.).

Expressed protein may be accumulated within a cell or may be secreted from the cell. Such expressed protein may then be collected and purified. This protein may then be characterized for activity and heat stability and may be used to practice any of the various methods according to the disclosure.

E. Isolation and Purification of Mannosidase 1A, 1B, and 1C

The mannosidases of the present disclosure can be isolated and purified from either transgenic host cells, or from wild-type host cells. The purification of the mannosidase enzymes of the present disclosure is achieved by first isolating the enzymes from the other cellular components and then purifying the enzymes.

1. Separation of Enzymes from Cellular Components

A variety of methods are known in the art for separating enzymes from other cellular components (complexes). Such methods can involve treatment with harsh chemicals and/or severe temperatures. Typically, protein complexes can be disrupted through the use of reducing agents, denaturants, freeze/thaw cycles, mechanical shearing, decompression/compression, sonication, agitation, and/or increased temperatures. Protein complexes also can be disrupted using a combination of such techniques.

Reducing agents are capable of donating hydrogen atoms, and thus serve to cleave disulfide bonds. Reducing agents particularly disrupt disulfide bonds that link two proteins or portions of the same protein together. Commonly used reducing regents are P-mercaptoethanol, dithiothreitol (DTT), and trialkyl phosphines. The ability of a reducing agent to disrupt a complex is increased by increasing the temperature at which a mixture of the reducing agent and complex is incubated.

Denaturants serve to relax the conformational structure of proteins. Any of various denaturants can be employed to separate a protein from other cellular components. Examples of denaturants useful in conjunction with ion-exchange columns are urea and formamide. However, denaturants such as guanidine hydrochloride or guanidine thiocyanate may be useful for separating proteins from other cellular components in methods not involving an ion-exchange column. The ability of a particular denaturant to relax a protein is enhanced by increasing the temperature at which the sample is incubated.

2. Purification of Enzymes from Cellular Components

Purification of enzymes from a crude lysate can be achieved by successive rounds of chromatography. In between each round of chromatography, the sample is assayed for enzymatic activity. Examples 7 and 8, below, describe activity assays that can be used to monitor the recovery of mannosidase activity, respectively.

Typically, purification involves a multi-step procedure that includes well-known chromatographic techniques (Robyt and White, Biochemical Techniques Theory and Practice, Waveland Press, Inc., 1990). In such multi-step procedures, the enzymes are separated by exploiting their different physical characteristics. The following discussion provides a broad description of various chromatography techniques that can be used in either a single-step process or in a multi-step process. A single type of chromatography can be repeatedly used (rather than changing the chromatography step each time) to purify the enzymes of the present disclosure.

i. Column Chromatography

One method of isolating an enzyme according to the present disclosure is adsorption chromatography. This method exploits a protein's differential affinity for the medium in a column, compared to the protein's affinity for the eluting solvent.

An example of a suitable medium for use in the column is hydroxyapatite (crystalline calcium phosphate). Hydroxyapatite tends to adsorb acidic proteins that can be subsequently eluted with phosphate ions (phosphate ions have a high affinity for the calcium ions present in the hydroxyapatite).

Ion-exchange chromatography (a variation of adsorption chromatography) can also be used to isolate enzymes according to the present disclosure. In ion-exchange chromatography, a solid adsorbent is used that has charged groups chemically linked to an inert solid. An ionic charge on an enzyme molecule causes the molecule to attach to an oppositely charged group on the solid support. The enzyme is subsequently released from the support by passing a solution containing an ion gradient over the solid adsorbent. Examples of solid supports are DEAE-cellulose, DEAE-SEPHADEX™, DEAE-BIO-GEL™, DEAE-SEPHAROSE™, DEAE-SEPHACRYL™, DEAE-TRISACRYL™, Q-SEPHAROSE™, ecteola cellulose, QAE-cellulose, express ion exchanger Q, PEI-cellulose, and other polystyrene-based anion exchangers (most of these support materials are available from Pharmacia).

Another suitable type of adsorption chromatography is affinity chromatography. This method involves covalently linking to an inert solid support a ligand having a binding affinity for the subject enzyme. Commonly, the ligand is a specific binding agent that selectively binds the enzyme as the enzyme molecule contacts the solid support. Alternatively, the enzymes can be purified based upon their hydrophobicity. For example, alkyl chains can be linked to the inert support to supply sites for hydrophobic bonding interactions between the support and the enzyme. The eluting solvent contains a hydrophobic gradient.

High-performance liquid chromatography (HPLC) is yet another suitable method. All of the major classes of chromatographic separations are compatible with this method, for example: adsorption, liquid-liquid partition, ion exchange, exclusion, and affinity chromatography can be used in conjunction with HPLC.

Additionally, HPLC allows for reverse-phase and ion-pair partition. Reverse-phase partition is a relatively quick elution technique using a non-polar stationary phase and a polar mobile phase. Ion-pair partition involves pairing a charged polar substance with its counter-ion to create a less polar species that then flows through the column.

ii. Electrophoresis

Electrophoresis is a well-established technique for the separation and analysis of mixtures by differential migration and separation of molecules in an electric field, based on differences in mobility of the molecule through a support.

Many different forms of electrophoresis have been developed to permit the separation of different classes of compounds. These forms include paper and cellulose acetate electrophoresis, thin-layer electrophoresis, gel electrophoresis, immunoelectrophoresis, and isoelectric focusing. Paper electrophoresis operates best for the separation of protein molecules having a relatively low molecular weight, and gel electrophoresis is better for use in isolating enzymes with higher molecular weights. A variety of different matrices are available for forming gels, but matrices with a relatively small pore size are most suitable for the separation of two proteins that have similar physical characteristics. Furthermore, proteins may be separated based upon their molecular size by conducting electrophoresis under dissociating conditions, for example using sodium dodecyl sulfate (SDS). SDS relaxes the protein conformation and masks the ionic charge of the protein, hereby leaving the relative length of the protein as the principal distinguishing characteristic for purposes of separation from other proteins.

Separation also can be achieved using immunoelectrophoresis, in which proteins are separated on a gel, based upon the relative charge-to-mass ratio and antigenicity of the proteins. The proteins are separated on a gel. An antibody, specific for the protein of interest, is added to a well created in the gel, and the antibody is allowed to diffuse through the gel. A precipitate forms in regions in which the antibody reacts with the protein.

Finally, the enzymes can be purified by isoelectric focusing (IEF). IEF is a type of electrophoresis in which the protein is placed on a substrate having a pH gradient. The protein moves under the influence of an applied electrical field until the protein reaches a zone in the pH gradient corresponding to the isoelectric point of the protein.

Specific examples of previously published mannosidase-purification techniques are available in Castellion and Bretthauer, Biochem. J., 324:951-956, 1997; and Eades et al., Glycobiology 8:17-33,1998.

III. Mannosidase Specific Antibodies

Antibodies to the mannosidase enzymes of the present disclosure may be useful for purification of the enzymes. The provision of the amino acid sequences of the mannosidase 1 A, 1 B, and 1 C enzymes allows for the production of specific antibody-based binding agents to these enzymes.

A. Production of an Antibodies to Mannosidase 1A, 1B and/or 1C

Monoclonal or polyclonal antibodies may be produced to either of the mannosidase enzymes, portions of the enzymes, or variants thereof. Optimally, antibodies raised against epitopes on these antigens will specifically detect the enzyme. That is, antibodies raised against the enzymes recognize and bind the enzymes, but do not substantially recognize or bind to other proteins. The determination that an antibody specifically binds to an antigen is made by any one of a number of standard immunoassay methods, for instance, Western blotting, Sambrook et al. (ed.), Molecular Cloning: A Laboratory Manual, 2nd ed., vol. 1-3, Cold Spring Harbor Laboratory Press, Cold Spring Harbor, N.Y., 1989. To determine that a given antibody preparation (such as a preparation produced in a mouse against mannosidase 1 A) specifically detects a mannosidase by Western blotting, total cellular protein is extracted from fungal cells and electrophoresed on an SDS-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase; application of 5-bromo-4-chloro-3-indolyl phosphate/nitro blue tetrazolium results in the production of a dense blue compound by immuno-localized alkaline phosphatase.

Antibodies that specifically detect mannosidase can be shown, by this technique, to bind substantially only the mannosidase band (having a position on the gel determined by the molecular weight of the mannosidase). Non-specific binding of the antibody to other proteins may occur and may be detectable as a weaker signal on the Western blot (which can be quantified by automated radiography). The nonspecific nature of this binding can be recognized by one skilled in the art by the weak signal obtained on the Western blot relative to the strong primary signal arising from the specific anti-mannosidase binding.

Antibodies that specifically bind to mannosidase belong to a class of molecules that are referred to herein as “specific binding agents.” Specific binding agents that are capable of specifically binding to the mannosidases of the present disclosure may include polyclonal antibodies, monoclonal antibodies, and fragments of monoclonal antibodies such as Fab, F (ab′) 2 and Fv fragments, as well as any other agent capable of specifically binding to one or more epitopes on the proteins.

Substantially pure mannosidase 1A or 1B suitable for use as an immunogen can be isolated from transfected cells, transformed cells, or from wild-type cells.

Concentration of protein in the final preparation is adjusted, for example, by concentration on an Amicon filter device (Charlotte, N.C.), to the level of a few micrograms per milliliter. Alternatively, peptide fragments of either of the mannosidases may be utilized as immunogens. Such fragments may be chemically synthesized using standard methods, or may be obtained by cleavage of the whole mannosidase enzyme followed by purification of the desired peptide fragments.

Peptides as short as three or four amino acids in length are immunogenic when presented to an immune system in the context of a Major Histocompatibility Complex (MHC) molecule, such as MHC class I or MHC class II. Accordingly, peptides comprising at least 3 and preferably at least 4,5,6 or more consecutive amino acids of the disclosed mannosidase amino acid sequences may be employed as immunogens for producing antibodies. Because naturally occurring epitopes on proteins frequently comprise amino acid residues that are not adjacently arranged in the peptide when the peptide sequence is viewed as a linear molecule, it may be advantageous to utilize longer peptide fragments from the mannosidase amino acid sequences for producing antibodies. Thus, for example, peptides that comprise at least 10,15,20,25, or 30 consecutive amino acid residues of the amino acid sequence may be employed. Monoclonal or polyclonal antibodies to the intact mannosidase, or peptide fragments thereof may be prepared as described below.

B. Monoclonal Antibody Production by Hybridoma Fusion

Monoclonal antibody to any of various epitopes of the mannosidase enzymes that are identified and isolated as described herein, can be prepared from murine hybridomas according to the classical method of Kohler & Milstein, Nature, 256: 495,1975, or a derivative method thereof. Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, Enzymol. 70: 419, 1980, or a derivative method thereof. Selected positive clones can be expanded and their monoclonal antibody product harvested for use. Detailed procedures for monoclonal antibody production are described in Harlow & Lane, Antibodies, A Laboratory Manual, Cold Spring Harbor Laboratory, New York, 1988.

C. Polyclonal Antibody Production by Immunization

Polyclonal antiserum containing antibodies to heterogenous epitopes of a single protein can be prepared by immunizing suitable animals with the expressed protein, which can be unmodified or modified, to enhance immunogenicity. Effective polyclonal antibody production is affected by many factors related both to the antigen and the host species. For example, small molecules tend to be less immunogenic than other molecules and may require the use of carriers and an adjuvant. Also, host animals vary in response to site of inoculations and dose, with both inadequate or excessive doses of antigen resulting in low-titer antisera. Small doses (ng level) of antigen administered at multiple intradermal sites appear to be most reliable. An effective immunization protocol for rabbits can be found in Vaitukaitis et al., J. Clin. Endocrinol. Metab. 33: 988-991,1971.

Booster injections can be given at regular intervals, and antiserum harvested when the antibody titer thereof, as determined semi-quantitatively, for example, by double immunodiffusion in agar against known concentrations of the antigen, begins to fall. See, for example, Ouchterlony et al., Handbook of Experimental Immunology, Wier, D. (ed.), Chapter 19, Blackwell, 1973. A plateau concentration of antibody is usually in the range of 0.

D. Antibodies Raised by Injection of cDNA

Antibodies may be raised against the mannosidases of the present disclosure by subcutaneous injection of a DNA vector that expresses the enzymes in laboratory animals, such as mice. Delivery of the recombinant vector into the animals may be achieved using a hand-held form of the Biolistic system (Sanford et al., Particulate Sci. Technol. 5: 27-37,1987, as described by Tang et al., Nature (London) 356: 153154,1992). Expression vectors suitable for this purpose may include those that express the cDNA of the enzyme under the transcriptional control of either the human P-actin promoter or the cytomegalovirus (CMV) promoter. Methods of administering naked DNA to animals in a manner resulting in expression of the DNA in the body of the animal are well known and are described, for example, in U.S. Pat. No. 5,620,896 (“DNA vaccines against rotavirus infections”); U.S. Pat. No. 5,643,578 (“Immunization by inoculation of DNA transcription unit”); and U.S. Pat. No. 5,593,972 (“Genetic immunization”), and references cited therein.

E. Antibody Fragments

Antibody fragments may be used in place of whole antibodies and may be readily expressed in prokaryotic host cells. Methods of making and using immunologically effective portions of monoclonal antibodies, also referred to as “antibody fragments,” are well known and include those described in Better & Horowitz, Methods Enzymol., 178: 476-496,1989; Better et al., in Streilein et al., eds., Advances in Gene Technology : The Molecular Biology of Immune Disease & the Immuneresponse (ICSUShortReports), 10:105, 1990;Glockshuber et al. Biochemistry 29: 1362-1367,1990; and U.S. Pat. No. 5,648,237 (“Expression of Functional Antibody Fragments”), U.S. Pat. No. 4,946,778 (“Single Polypeptide Chain Binding Molecules”), and U.S. Pat. No. 5,455,030 (“Immunotherapy Using Single Chain Polypeptide Binding Molecules”), and references cited therein.

IV. Using Mannosidase

A. In vitro

The mannosidases of the present disclosure are useful for modifying the glycosylation pattern proteins in vitro. In particular, the enzymes of the present disclosure are useful for modifying target proteins. Target proteins are specific proteins that are the desired product of a process, for example, a glycoprotein that will be used as a therapeutic agent.

The in vitro modification of a target protein is accomplished by first obtaining one or more of the mannosidases of the present disclosure. The mannosidase may be purified to achieve specificity of action. The mannosidase is then placed in contact with the target protein, allowing for the modification of the glycosylation pattern of the target protein in a controlled, in vitro environment. In vitro methods of modifying the glycosylation pattern of a protein are well known in the art, and examples of such methods are provided in U.S. Pat. No. 5,834,251 to Maras, et al., herein incorporated by reference.

B. In vivo

The glycosylation pattern of a protein, a broad class of proteins, or all glycosylated proteins in a host cell can be modified in vivo through the use of molecular biology techniques. The provision of the disclosed nucleic acid sequences allows for both the upregulation of the disclosed mannosides as well as the down-regulation of the disclosed mannosidases. Up regulation of the protein can be accomplished by transforming a cell with a vector containing one or more of the disclosed mannosidase genes under the control of a promoter. The promoter can be for example a constitutive promoter, a tissue specific promoter, or an inducible promoter. Thus, the RNA encoding the enzyme (s) is produced and translated into the protein. The increased level of mannosidase in the cell will then alter the glycosylation pattern of proteins produced by the cell. Conversely, the production of mannosidase by a cell can be down-regulated by transforming the cell with a vector encoding an antisense molecule, or a catalytic nucleic acid molecule that targets the mannosidase specific nucleic acid sequences. This technique will likely cause the cell to produce less mannosidase and therefore, glycoproteins from the cell will be processed to a lesser extent.

In contrast to the above-described in vitro methods, in vivo methods can be used to produce multiple different proteins in the same cell, all of which display altered glycosylation patterns.

### V. EXAMPLES

### Example 1

Identification of Aspergillus nidulans α-1,2-mannosidase 1A. Four previously published Class 1 α-1, 2-mannosidase protein sequences were aligned, and conserved blocks were identified for PCR primer design. Selection of sequences for primer design was based on high sequence identity between the four proteins, and low codon redundancy. A codon-preference table for A. nidulans was used to decrease the codon redundancy in the primers and to select codons more likely to be found in the genomic DNA. Two primers were selected that contained the least redundancy and the highest possible annealing temperature. Amplification of A. nidulans genomic DNA with these two primers yielded a 900-bp product. This PCR product was cloned and the DNA sequence was determined for several representative clones. The DNA sequence of clone pGEM42-9 was used to search the GenBank database for sequence homology (BLAST TM search). Several high scoring matches were found with other Class 1 α-1,2-mannosidases, confirming that a portion of the Class 1 α-1,2-mannosidase from A. nidulans had been amplified.

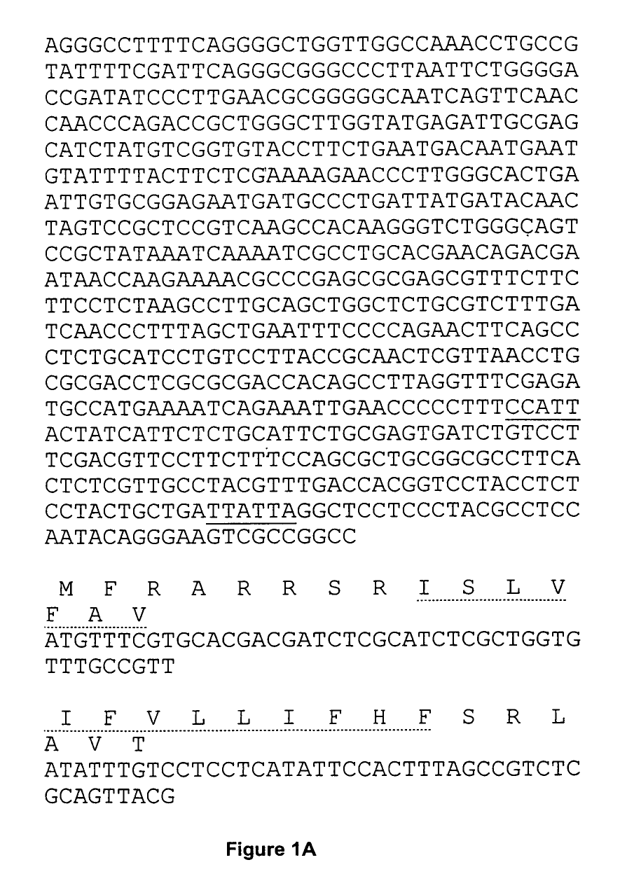

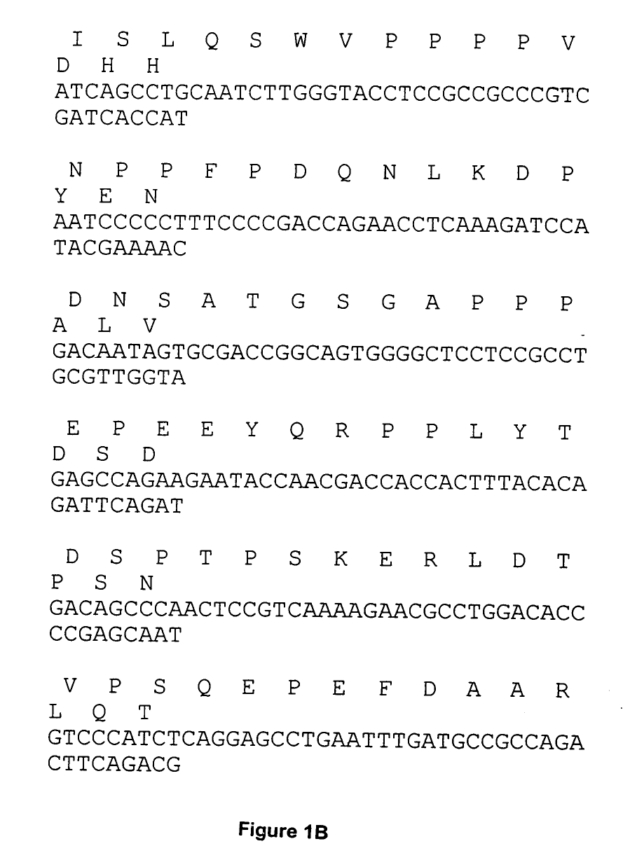

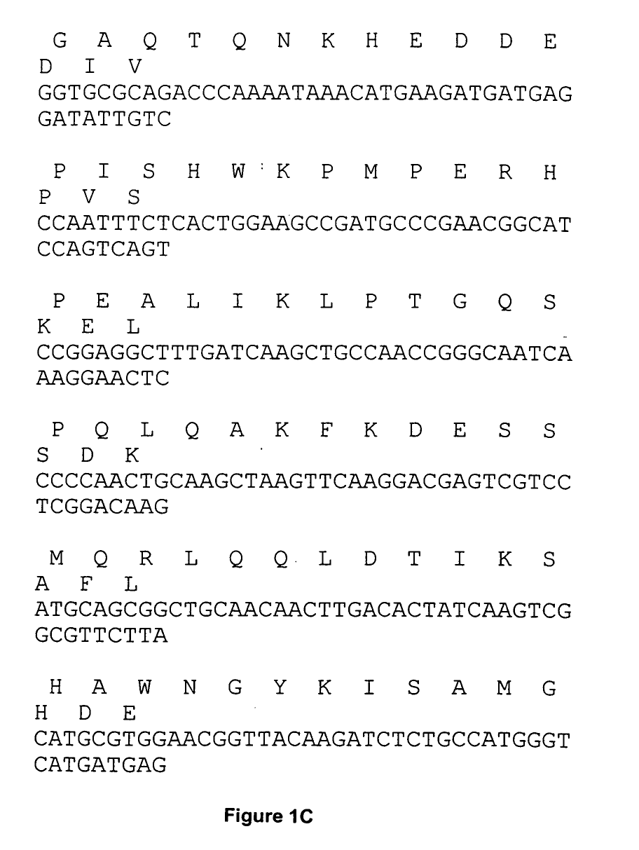

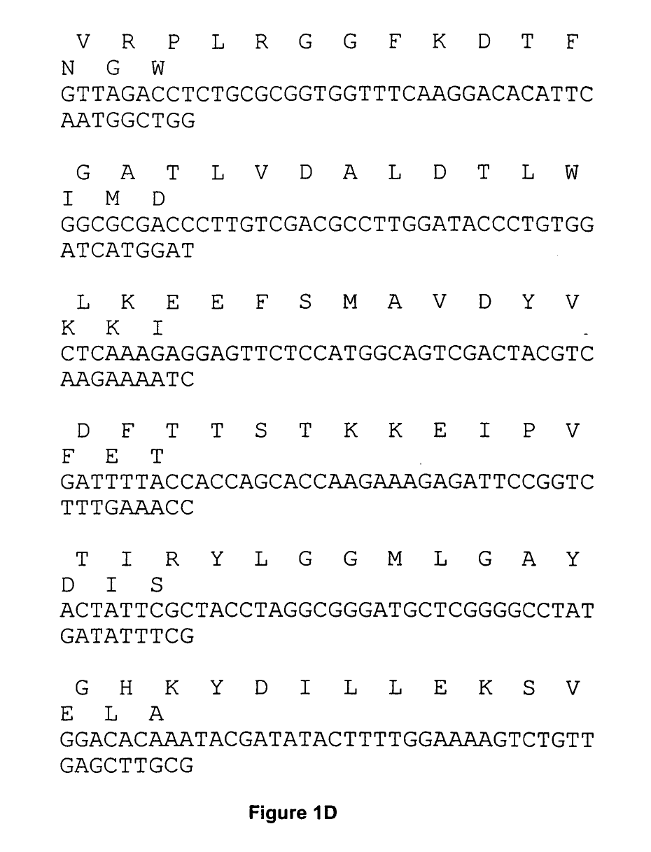

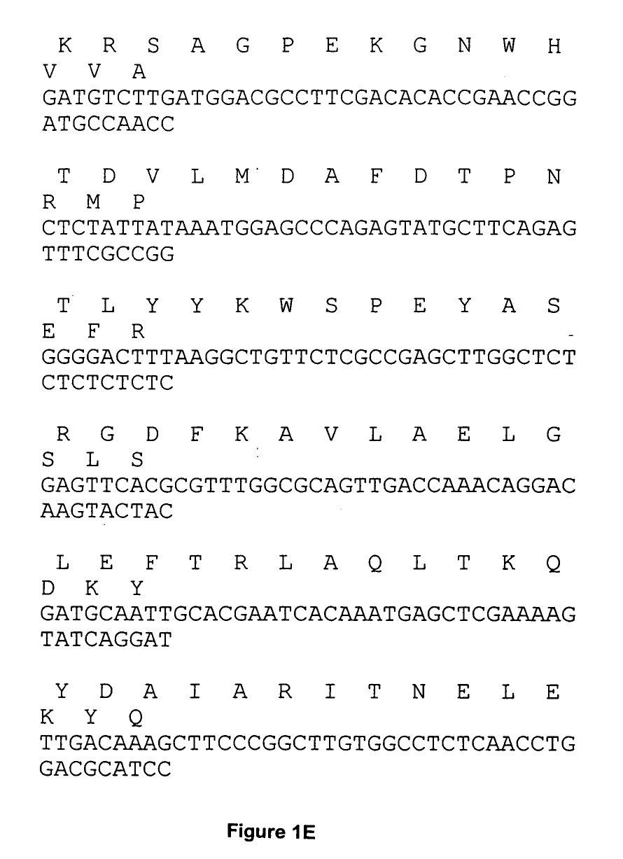

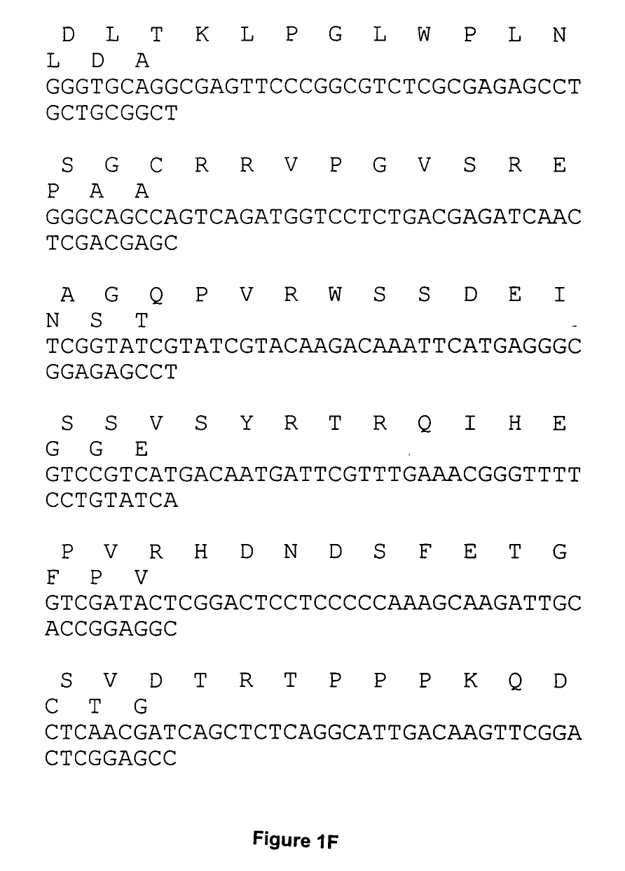

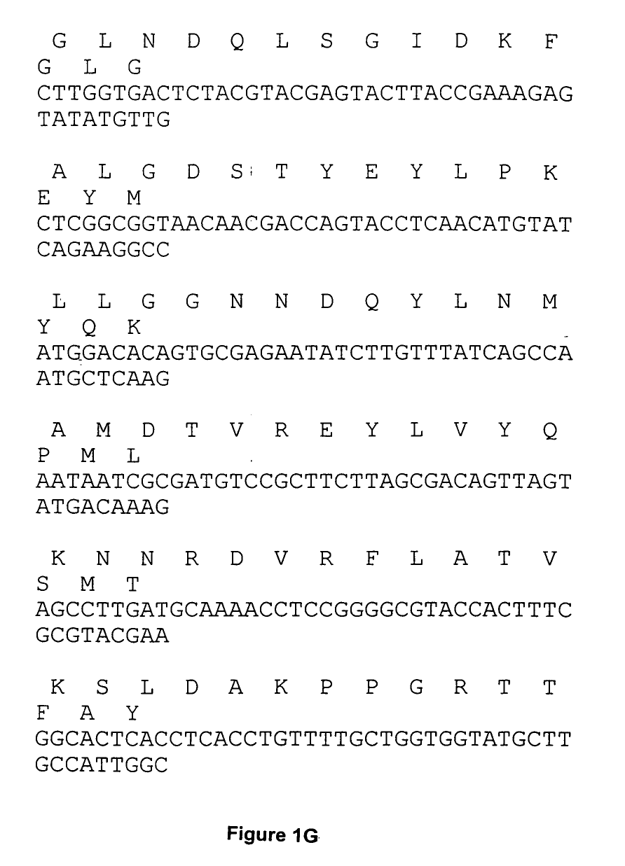

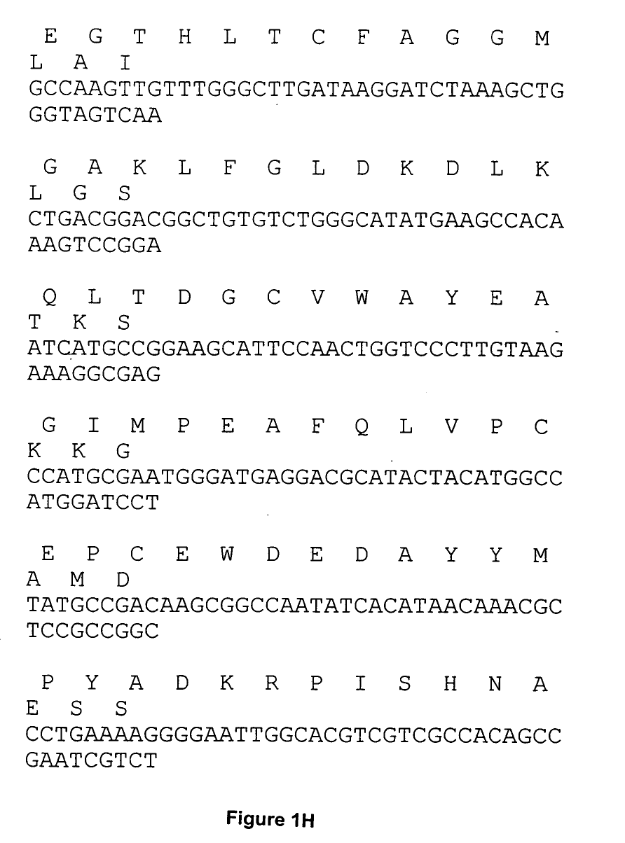

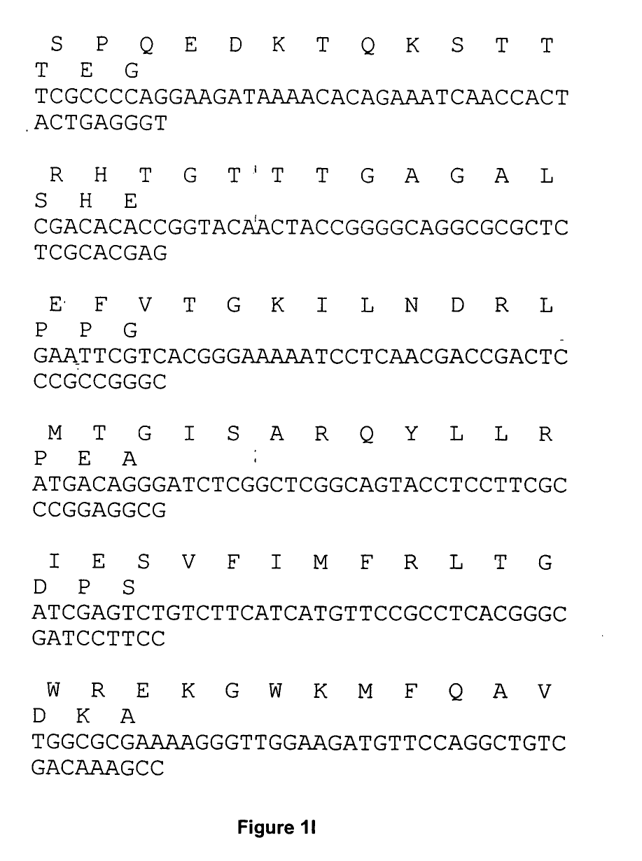

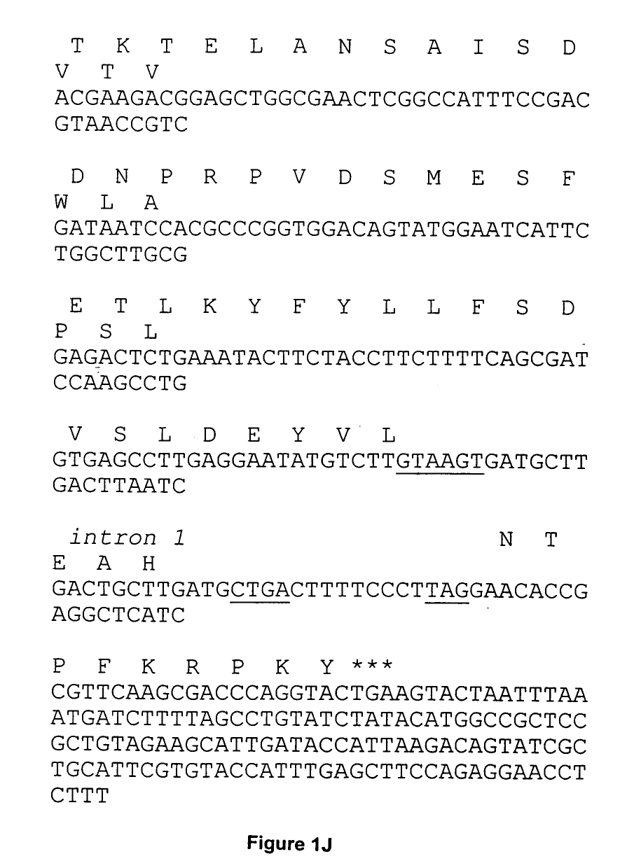

The 900-bp PCR fragment was used as a template for the PCR amplification of an α-1,2-mannosidase-specific radiolabeled probe for library screening. An EMBL-3 library of genomic sequences was screened and several positive plaques were re-screened to isolate a single lambda clone containing the entire α-1,2-mannosidase gene. Two BamH1 subclones were identified by Southern hybridization, using the same PCR-derived probe, that contained the 5′and 3′ends of the gene. These subclones were sequenced by manual and automated sequencing, using a combination of primer walking with specifically designed primers and restriction enzyme subcloning using universal sequencing primers, yielding 2- to 5 fold redundancy sequencing of the full-length gene and several thousand base pairs (bp) of flanking sequence (FIG. 1).

### Example 2

Characterization of the α-1,2-mannosidase 1A gene. A BLAST search of the α-1,2-mannosidase 1A gene revealed two open reading frames (ORFs) that contained significant homology to the Class 1 α-mannosidases. These ORFs were separated by a region of DNA containing several stop codons, which indicated an intron sequence. This intron sequence was verified by reverse-transcription PCR (RT-PCR) from total RNA and comparison to the genomic DNA. The amplification product derived from the RNA, using a PCR primer pair that flanked the putative intron, was 50 bp shorter than the similar PCR product derived from genomic DNA.

Analysis of the putative intron revealed several typical sequence motifs which are characteristic of eukaryotic intron sequences. The 5′-splice site (5′-GTAAGT-3′ (SEQ ID NO: 7)) fit the consensus sequence for filamentous fungi (5′-GTANGT-3′ (SEQ ID NO: 8)), and the 3′-splice site (5′-TAG-3′) fit the consensus 5′-YAG-3′ (Ballance, Molecular Industrial Mycology-Systems and Applications for Filamentious Fungi, Leon and Berka (eds.), Dekker, Inc., 1991; Ballance, Yeast 2: 229-236,1986; Gurr et al., Gene Structure in Eukarayotic Microbes, Kinghorn (ed.), IRL Press, 1987). The intron also contained an internal lariat sequence (5′ GCTGAC-3′ (SEQ ID NO: 9)), located 15 bp upstream of the 3′-splice site, which fit the consensus5′-(G/A) CT (G/A) AC-3′ (SEQ ID NO: 19).

The deduced amino acid sequence of the A. nidulans α-1,2-mannosidase 1A gene was aligned with other published a-mannosidases in order to identify potential introns that do not shift the reading frame and do not contain stop codons but do increase the size of the putative gene product. In addition to the previously confirmed intron, two other regions of the ORF that did not align with other published sequences seemed to be “extra” DNA sequences. These regions were represented by large “gaps” in the multiple sequence alignment for all of the other a-1, 2-mannosidase genes used in the alignment. To determine whether these sequences represented introns, or encoded a polypeptide sequence novel to the A. nidulans α-mannosidase, primer pairs were designed that flanked these regions and used in RT-PCR. The amplification products derived from the RNA (RT-PCR) were the same size as the amplification products derived from genomic DNA, indicating that there were no introns present in these regions. These regions did not contain consensus splice motifs, though sometimes introns were present with less conserved splice sequences (Gurr et al., Gene Structure in Eukarayotic Microbes, Kinghorn (ed.), IRL Press, 1987).

The position of the translational start codon of the first ORF was determined through examination of the DNA sequence near the stop codon that defined the 5′ end of the ORF. A potential in-frame start codon (ATG) occurred 42 bp after the beginning of the ORF, while the next in-frame ATG codon was located 423 bp downstream. The first ATG codon was thus a good candidate for the translational start codon. Translation originating at the first start codon would produce a protein product with an N-terminus larger than other fungal a-mannosidases, but similar in size to the N-termini of mammalian and insect Class 1 a-mannosidases.

Though no strong consensus sequence surrounds the translational start codon in filamentous fungi (Ballance, Yeast 2: 229-236,1986), there is a preference (97%) for a purine at position-3. The putative start codon for this gene has a G at position-3, and thus conforms to this rule. There is a TATA-like element at position-47 and several pyrimidine-rich blocks within 100 bp upstream of the putative start codon.

These pyrimidine rich regions are often found in fungal promoters and may influence the level of transcription (Ballance, Yeast 2: 229-236,1986). The 5′ untranslated region does not contain a CAAT-box upstream of the TATA-like element, but this is not unusual for fungal promoters. The α-1,2-mannosidase 1A protein contained several charged N-terminal amino acids representative of a typical signal sequence motif. A Kyte-Doolittle hydropathy plot showed that the signal sequence was followed by a highly hydrophobic region approximately 15-16 amino acids in length, likely encoding a transmembrane domain, while the rest of the protein was relatively hydrophilic (FIG. 3). This α-1,2-mannosidase 1A protein from A. nidulans likely forms a type II transmembrane protein, which is a characteristic of other Class 1 α-1,2 mannosidases.

### Example 3

Identification of A. nidulans α-1, 2-mannosidase 1B. The deduced coding region of the A. nidulans α-1,2-mannosidase 1A gene was used to search the A. nidulans EST Sequencing Project Database using the BLAST algorithm to determine if there were multiple α-1,2-mannosidase genes were expressed in this organism. A sequence tag, containing 200 bp of information, was identified that showed significant similarity, but not 100% identity, to the α-1,2-mannosidase 1A gene, and thus represented a separate gene. This sequence tag was used to design new PCR primers specific for the novel gene (α-1,2-mannosidase 1B). The primers were designed in regions that, based on multiple sequence alignments of published α-1,2-mannosidase sequences, would not be expected to be well conserved, and thus would be gene-specific. The 200-bp amplification product from these primers was radiolabeled and used to probe the A. nidulans genomic library to select the full length gene.

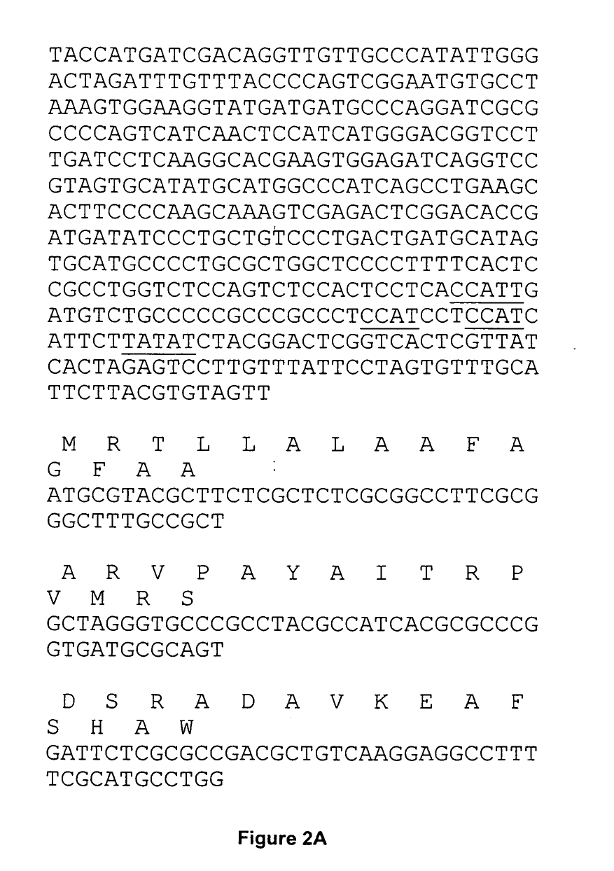

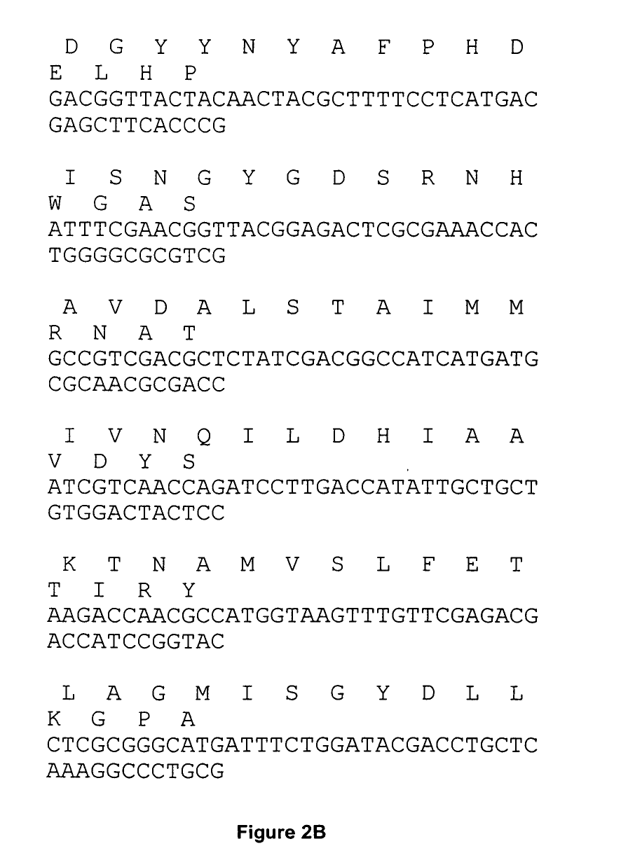

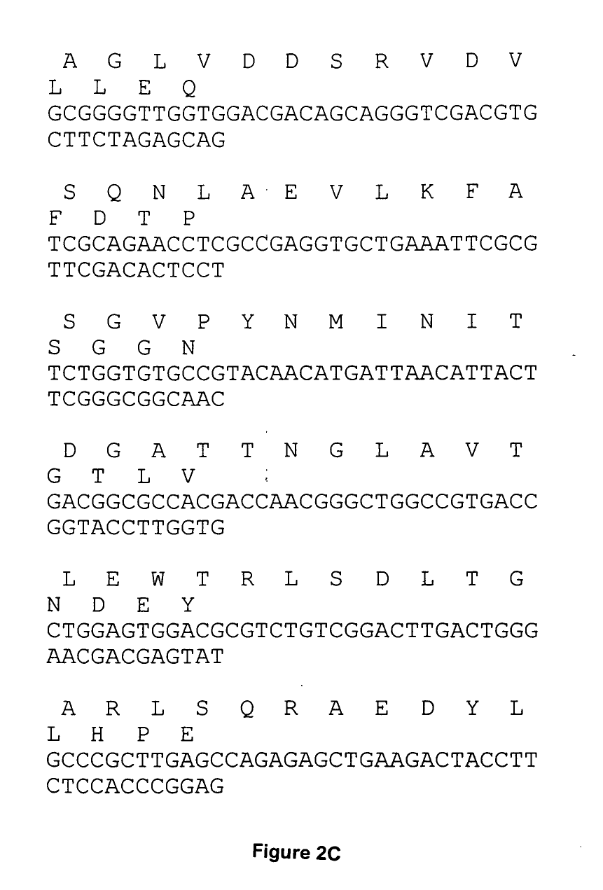

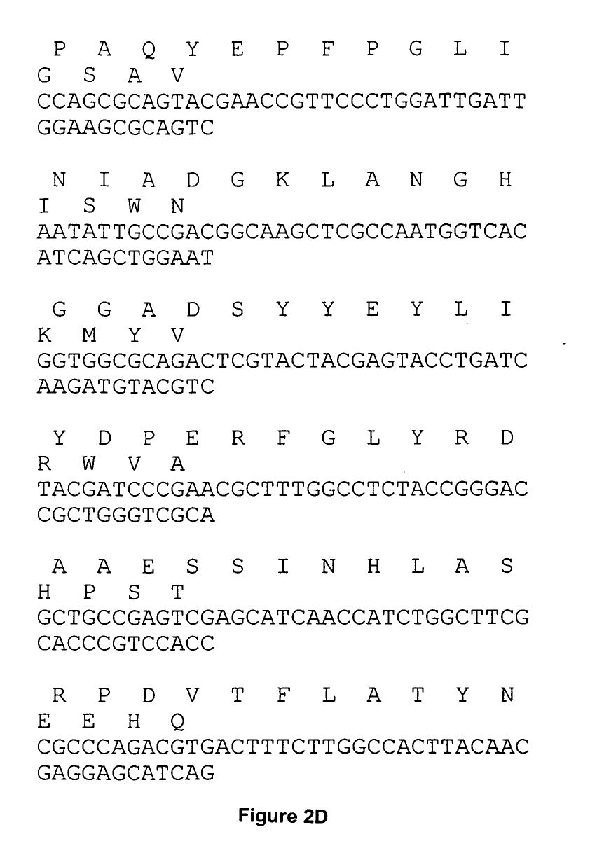

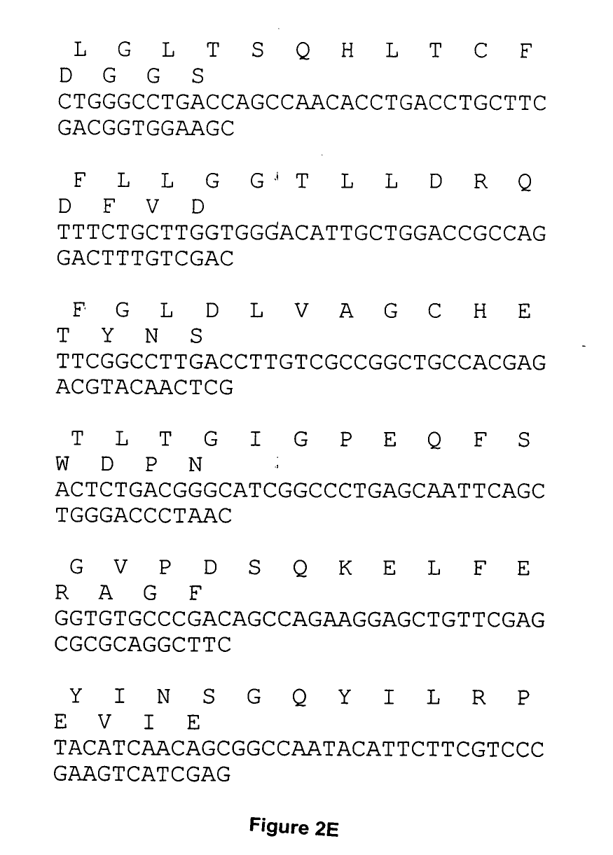

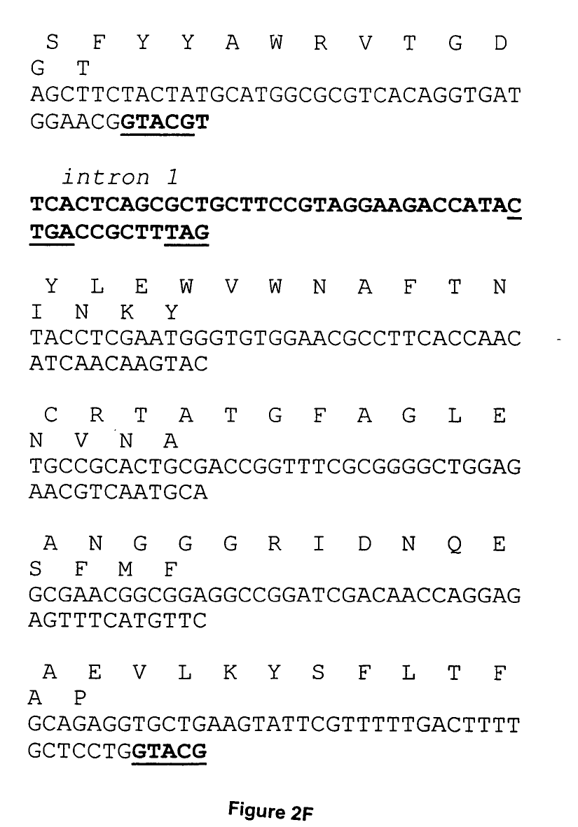

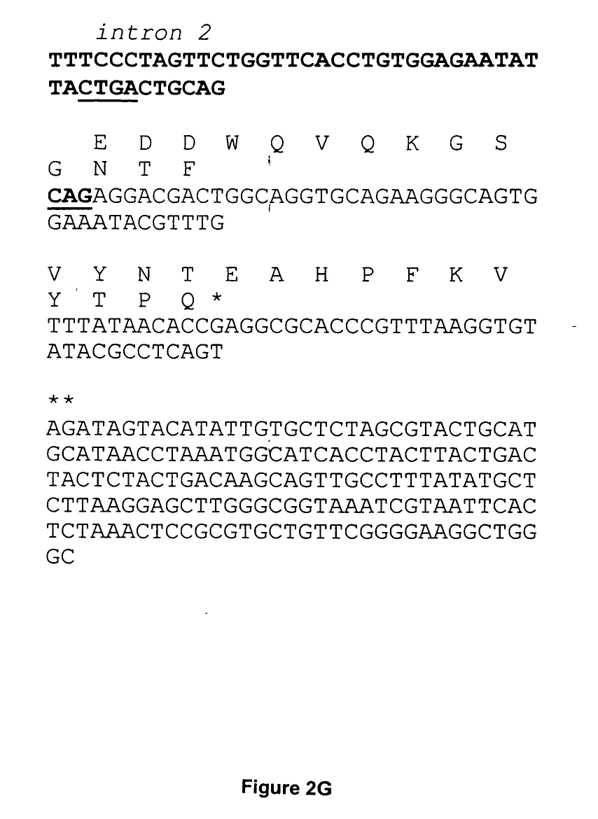

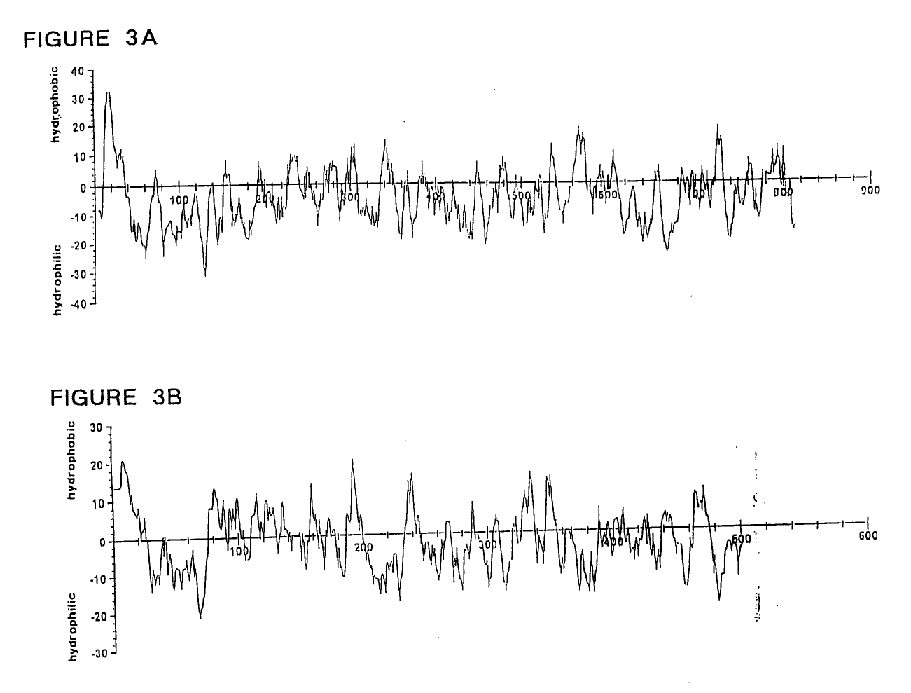

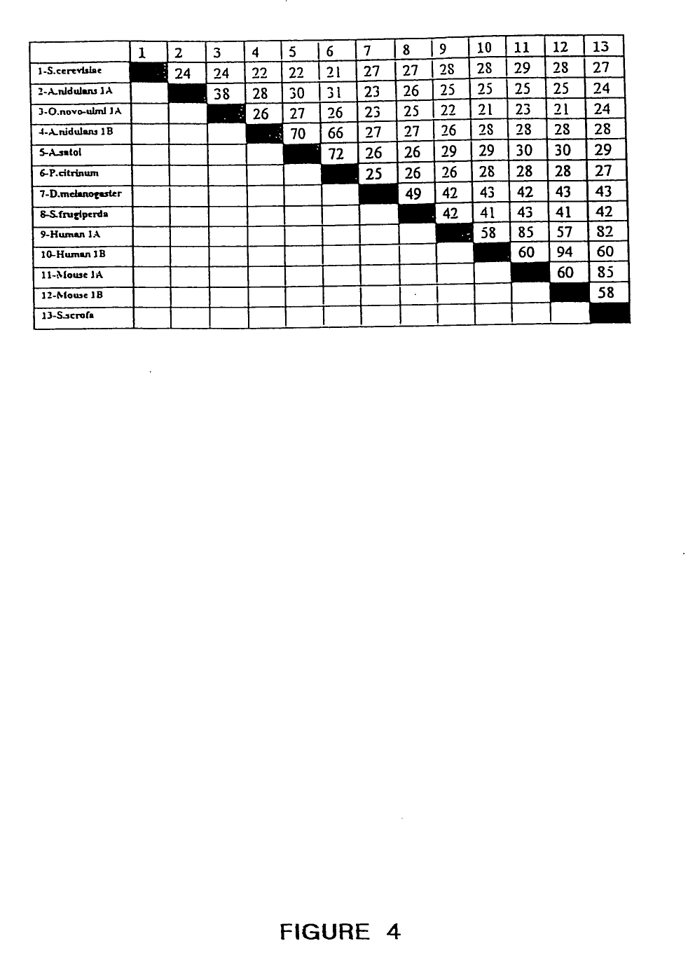

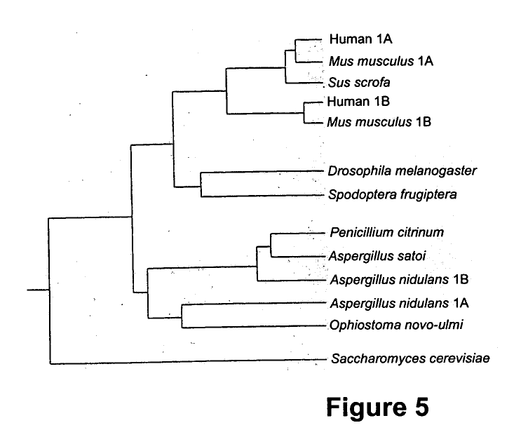

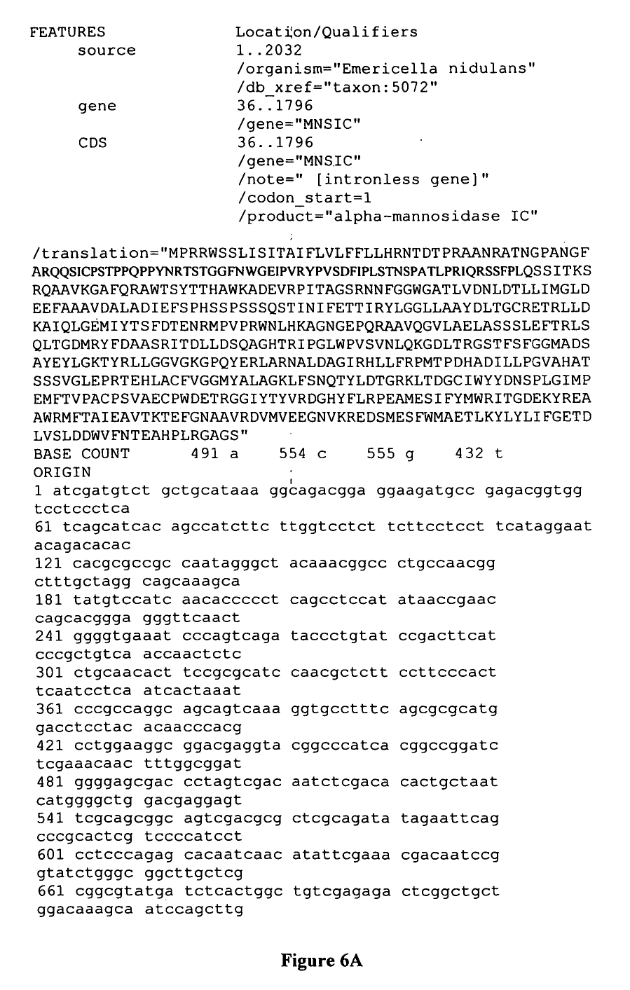

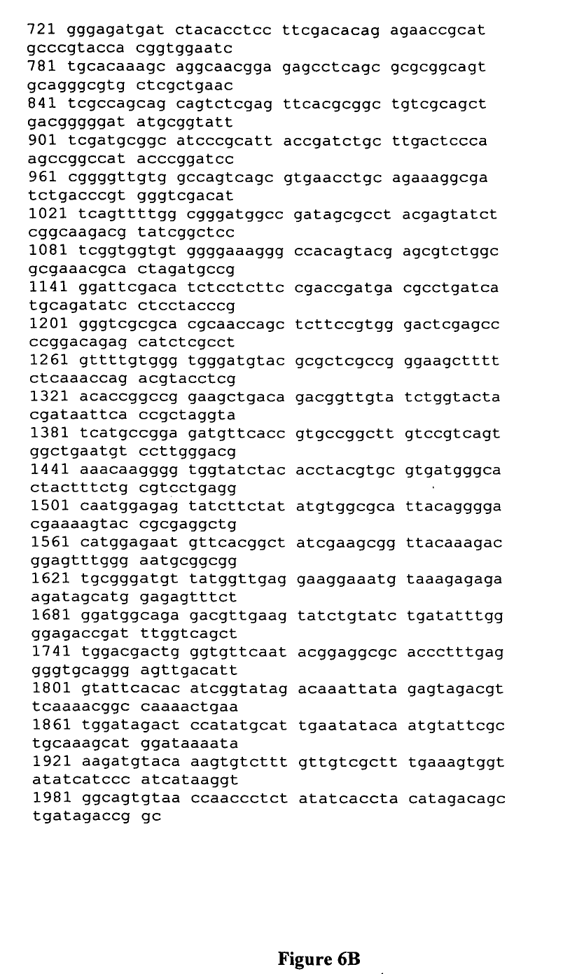

A single lambda clone containing the full-length α-1,2-mannosidase 1B gene was isolated and subcloned into plasmid DNA. A single 5.6-kb BamHI clone containing the full-length gene was identified by Southern analysis. A series of restriction enzyme resections of the 5.6-kb clone were derived and the sequence was determined by automated fluorescence sequencing using the universal priming sites of the cloning vector. The full-length gene and several hundred bases of flanking sequence were sequenced with 2- to 4-fold redundancy at each base position (FIG. 2).

### Example 4

Characterization of α-1, 2-mannosidase 1B. The genomic DNA sequence for the A. nidulans α-1,2-mannosidase 1B gene was searched for open reading frames that showed homology to other α-1,2-mannosidase genes. The gene contained three open reading frames with homology with Class 1 α-1,2 mannosidases separated by two regions that contained several stop codons and caused a shift in the reading frame containing α-1, 2-mannosidase homology. The two regions that disrupted the open reading frame were analyzed to identify potential introns. Comparison of the reading frame of the gene with the A. satoi and P. citrinum genes verified that these regions appeared to contain extra DNA sequences, since the alignment seems to be disrupted near these regions. These regions were searched for consensus intron-splice sites. The first potential intron did indeed contain 5′and 3′consensus splice sites as well as an internal lariat consensus sequence. The sequence 5′-GTACGT-3′ (SEQ ID NO: 11) fit the filamentous fungal consensus for a 5′-splice site (5′-GTANGT-3′), and the sequence 5′-TAG-3′fit the consensus for the 3′-splice site (5′-YAG-3′). This intron also contained the internal lariat sequence 5′-ACTGAC-3′ (SEQ ID NO: 12) located 11 bp upstream of the 3′-splice site. The second putative intron also contained a consensus 5′-splice site (5′-GTACGT-3′), a consensus 3′-splice site (5′-CAG-3′), and an internal lariat consensus (5′-ACTGAC-3′) which was located 11 bp upstream of the 3′-splice site. The two putative introns were verified by RT-PCR. In both cases, the RNA-derived amplification product was smaller than the DNA-derived amplification product, and the size difference corresponded to the predicted size of each intron. Interestingly, the position of each intron in the coding region is correlated directly to two of the introns found in the P. citrinum α-1,2-mannosidase gene, although the intron sequences themselves did not appear to be conserved. The N-terminal region of the first ORF likely contained the translational start site, which would probably be the first methionine after the stop codon defining the beginning of the ORF. Comparison of the coding region of this ORF with the A. satoi and P. citrinum α-1,2-mannosidase genes showed that the putative translational start site correlated positionally with the start sites of these genes. This start codon contained a purine at the −3 position, as expected (Ballance, Yeast 2: 229-236,1986). A TATA-like element was found at position-76 (TATAT), and the upstream region contained several CT-rich tracts in the sense strand. Seven copies of the sequence CTCC appeared in the 100-bp region upstream of the TATA-like element and could represent an important promoter element. Additionally, there was a 7-bp direct repeat located immediately upstream of the TATA-like element (CCTCAT). The N terminus of this protein contained a typical signal sequence for insertion into the endoplasmic reticulum and, as seen in a Kyte-Doolittle hydropathy plot (FIG. 3), contained a 10-15 bp hydrophobic region which likely encoded a transmembrane domain. This protein, like other Class 1 α-1,2-mannosidases, is likely a type-11 transmembrane protein normally localized in the ER or Golgi apparatus.

### Example 5

Identification of A. nidulans α-1,2-mannosidase IC: A second lambda clone containing the full lengtha-1, 2-mannosidase 1C gene was recovered and two non-overlapping BamHI subclones (4 kb and 6 kb) were isolated, which together contained the gene and flanking regions. Again, the sequence across the BamHI subcloning junction was verified to eliminate the possibility of missing sequence.

The gene and several hundred bp of flanking region were fully sequenced (Accession#: AF233287).

### Example 6

Comparison of α-1,2-mannosidase 1A, 1B, and 1C sequences: The DNA sequences of the three Class I α-1, 2-mannosidases were analyzed to determine the amino acid coding sequence, including determination of the correct reading frame, identification of potential intron sequences, and identification of the correct translational start codon for each gene. A BLAST search of the α-1,2-mannosidase IA gene revealed two open reading frames(ORFs) separated by a region of DNA containing several stop codons which could indicate the presence of an intron sequence. To verify the presence of an intron at this site, PCR products spanning the putative intron were amplified from reverse-transcribed RNA (RT-PCR), cloned into vector and sequenced. Comparison of the a-mannosidase IA sequence with RT PCR sequence verified the presence of a 50 bp intron at the expected splice junction. The intron contained a 5′-splice site (5′-GTAAGT-3′) which matched the consensus sequence for filamentous fungi (5′-GTANGT-3′), and a 3′-splice site (5′-TAG-3′) which matched the consensus 5′-YAG-3′ (Ballance, Transformation Systems for Filamentous Fungi and an Overview of Fungal Gene Structure, In Leong, S. A. and Berka, R. M. (eds.), Molecular Industrial Mycology-Systems and Applications for Filamentous Fungi, Dekker, Inc., New York, N.Y., pp. 1-29,1991,1986; Gurr et al., The Structure and Organization of Nuclear Genes of Filamentous Fungi, In Kinghorn (ed.), Gene Structure in Eukaryotic Microbes, IRL Press, Oxford, Wash., pp. 93-139,1987). The intron also contained an internal lariat sequence (5′-GCTGA3′; SEQ ID NO: 9), located 15 bp upstream of the 3′-splice site, consistent with the consensus 5′-(G/A) CT (G/A)AC-3′ (SEQ ID NO: 19) for fungal introns.

The deduced amino acid sequence of the A. nidulans α-1,2-mannosidase 1A gene was aligned with other published a-mannosidases to determine whether there might have been other introns which did not shift the reading frame and did not contain stop codons but did increase the size of the putative gene product. Two additional regions of the first ORF did not align with other published sequences and appeared as large ‘gaps’ in the multiple sequence alignment for all of the other α-1,2-mannosidase genes used in the alignment. To determine whether these sequences represented introns or encoded polypeptide sequence novel to the A. nidulans a-mannosidase, RT-PCR products were compared to genomic PCR products. The PCR amplification products derived from both RNA and genomic DNA, using primer pairs which flanked these regions, were the same size, indicating that there were no introns present in these regions. Although introns can sometimes be present with less conserved splice sequences (Gurr et al., The Structure and Organization of Nuclear Genes of Filamentous Fungi, In Kinghorn (ed.), Gene Structure in Eukaryotic Microbes, IRL Press, Oxford, Wash., pp. 93-139,1987), these regions did not contain consensus splice motifs. Thea-1, 2-mannosidase 1B gene contained three open reading frames separated by two regions which contained several stop codons and caused a shift in the reading frame. These regions were searched for consensus intron splice sites. Both of the putative introns contained consensus 5′-and 3′-splice sites and a consensus internal lariat sequence. The two putative introns were verified by RT PCR. In both cases, the RNA-derived amplification product was smaller than the DNA-derived amplification product, and the size difference corresponded to the predicted size of each intron. Sequencing of the RT-PCR products verified the presence of a 51 bp and 53 bp intron at the respective splice sites. While the position of each intron in the coding region correlated directly to two of the introns found in the P. citrinum α-1,2-mannosidase gene, the intron sequences themselves were not conserved. The α-1,2-mannosidase 1C gene contained a single contiguous open reading frame and did not contain any consensus intron sequences. This gene did not appear to contain any intron sequences.

The authentic start codon (ATG) of the α-1,2-mannosidase 1A gene was inferred by sequence context of the putative start codons, combined with protein sequence alignments with known α-mannosidase proteins. A potential start codon (ATG) occurred in frame 42 bp after the stop codon which defined the 5′-end of the first ORF, while the next in frame ATG codon was 423 bp downstream. This first codon was thus a better candidate for the translational start codon. Translation originating at this start codon would produce a protein product with an N-terminus which was larger than other fungal α-mannosidases, but similar in size to the termini of mammalian and insect Class I α-mannosidases. Although there is not a strong consensus sequence surrounding the translational start codon in filamentous fungi (Ballance, Yeast 2: 229-236,1986), there is a preference for a purine at position-3 (97%). The putative start codon for this gene had a G at position-3, and thus conforms to this rule. Placement of the translational start codon can also be inferred from sequence context with respect to promoter elements and the transcription start site. There was a TATA-like element at position-47 of the putative start site and several pyrimidine rich blocks within 100 bp upstream of the proposed start codon.

These pyrimidine rich regions are often found in fungal promoters and may influence the level of transcription (Ballance, Yeast 2: 229-236,1986). The 5′nontranslated region did not contain a CAAT-box upstream of the TATA-like element, but this is not unusual for fungal promoters.

The first potential translational start codon in the α-1,2-mannosidase 1B gene occurred 42 bp into the first ORF. Comparison of the coding region of this ORF with the A. satoi and P. citrinum α-1,2-mannosidase genes showed that the position of the putative translational start site correlated with the start sites of these genes.

This start codon also contained a purine at the −3 position, a TATA-like element at position-76, and several CT-rich blocks in the sense strand. The first potential translational start codon of the a-mannosidasel C gene occurred 38 bp into the ORF. The start site also contains a purine at position-3 and CCAAT motifat-221, but did not contain a clearly definable TATA box.

The putative coding region of the α-1,2-mannosidase 1A gene encodes a 816 amino acid protein with a predicted molecular weight (MW) of 91 kD. This is somewhat larger than other Class I α-mannosidases, which range in size from 53 kD for the P. citrinum α-mannosidase I (Yoshida et al., Biochim. Biophys. Acta 1263: 159-162,1995) to 73 kD for the H. α-mannosidase 1B (Bause et al., Eur. J. Biochem. 217:535-540, 1993), M. musculus α-mannosidase 1A (Lal et al., J. Biol. Chem. 269: 9872-9881,1994) and α-mannosidase 1B (Herscovics et al., J. Biol. Chem. 269: 9864-9871,1994), and S. scrofa α-mannosidase I (Bieberich et al., Eur. J. Biochem. 246: 681-689,1997). The coding region of the α-1,2-mannosidase 1B gene encodes a 505 aa protein with a predicted MW of 56 kD, while the α-1,2-mannosidase 1C gene encodes a 586 aa protein with a predicted MW of 65 kD.

Both of these predicted sizes are within the range of currently identified Class I α-mannosidases.

The putative α-1,2-mannosidase 1A, 1B, and 1C proteins contained several charged N-terminal amino acids representative of a typical signal sequence motif downstream of the putative start site. Kyte-Doolittle hydropathy plots showed that the signal sequences were followed by highly hydrophobic regions approximately 15-16 amino acids in length, while the remainder of the C-termini were relatively hydrophilic. These proteins likely form type II transmembrane proteins, which is a characteristic of other Class I α-1,2-mannosidases.

### Example 7

Mannosidase activity assays: Crude protein extracts were obtained from protoplasts and from culture filtrates. Secreted proteins were precipitated from 1.5 mL of culture filtrate by saturation with ammonium sulfate. After centrifugation, the protein extract was resuspended in 500 mL 0.01 M phosphate buffer (pH 6.0) and precipitated again using ammonium sulfate (to ensure removal of sugar residues which would interfere with mannosidase assays). Proteins were resuspended in 100 uL 0. 01 M phosphate buffer (pH6.0) and 27 uL of the extract was used in the mannosidase assay. Intracellular proteins were extracted from protoplasts, which were prepared as in Eades et al., Glycobiol. 8: 17-33,1998. Protoplasts were centrifuged and resuspended in 200 uL 0.1 M phosphate buffer (pH6.0)/1% octylthioglucoside, and resuspensions were vortexed vigorously to ensure complete protoplast lysis. Cellular debris was removed by centrifugation, and 27 uL of the lysate was used in mannosidase assays.

Mannosidase assays were performed using the disaccharideMan-a-1, 2-Maa-OCH3 as a substrate in a coupled enzyme assay as described earlier (Scaman et al., Glycobiol. 6: 265-270,1996), with some modifications. Digestion of the substrate was performed in a 300 uL final volume containing 270 uL of crude extract in 0.01M phosphate buffer (pH6.0) and 30 uL 100 mM disaccharide Man-α-1,2-Man-α-OCH3 incubated at 37 C for 3 hours. Detection of released mannose was achieved by addition of 30 uL Tris-HCl (pH7.6) and 240 uL of developing solution, containing glucose oxidase (55 U/mL), horseradish peroxidase(1 U/mL) and o-dianisidine dihydrochloride (70 ug/mL), incubated at 37 C for 3 hours. Absorbance measurements at 450 nm determined final color change. Standard blanks included all components of the colorimetric reaction, plus the substrate. As a control, enzyme extracts which were not used in the mannosidase digestion were subjected to the colorimetric reaction, to determine the absorbance which is due to the extract itself, and not due to mannose release. These values were subtracted from the absorbance values of the assays. Free mannose was used as a standard. All assays were performed in triplicate and the mean and standard deviation was calculated for each sample. Mannosidase activity was standardized by comparison with total protein in the crude enzyme extracts, and was de detergent buffer. Significant α-1,2-mannosidase activity was found in the intracellular protein extract (28.16 nmol mannose released from substrate/ug total protein/h; SD=4.45), whereas very little activity was found in the extracellular extracts (0.77 nmol/, ug/h ; SD=0.74). This is consistent with the hypothesis that the Class I α-1,2-mannosidase genes from A. nidulans encode type-II transmembrane proteins which are expressed intracellularly.

### Example 8

Enzyme and protein assays. Lal et al., Glycobiol. 8: 981-995,1998, described a protocol that can be used to assay for mannosidase activity. This protocol involved assays for testing the activity of two separate mannosidases.

Briefly, Lal et al., Glycobiol. 8: 981-995,1998, assayed for mannosidase activity using the disaccharide Man-α-1,2-Man-α-O-CH3, as a substrate and the glucoseoxidase/peroxidase coupled enzyme assay as described earlier (Scarnan et al., Glyco. Biol. 6: 265-270,1996) with some modifications. Assays testing mannosidase activity in crude culture medium, or purified enzyme preparations were performed in flat-bottomed 96-well microtiter plates in a 250-uL total reaction volume containing 0.1 M potassium phosphate, pH 6.0,210 mM Man-α-1, 2-Man-O-CH3 and 5-15 mL of enzyme solution at 37 C for 30 minutes, or as specified. The reaction was terminated by adding 25 uL of 1.25 M Tris-CL, pH 7.6.

The La et al., Glycobiol. 8: 981-995,1998, also provided an additional assay, that was used to test the activity of a second mannosidase. This second mannosidase was assayed in culture medium following 10-fold concentration using a Centricon 30 ultrafiltration membrane (Amicon). Assays were performed in 0.5 mL microfuge tubes in 30 uL total reaction volume containing 0.01 M potassium phosphate, pH 6.0, 210 mM Man α 1, 2Man-α-O-CH3, and 15-24 RL of concentrated culture medium at 37 C for 2 hours, or as specified. The reaction was terminated by heating to 100 C for 5 minutes followed by centrifugation at 16,000×g for 5 minutes. The supernatants (25 uL) were transferred to microtiter plate wells containing 25 uL of 1.25 M Tris-CL, pH 7.6. In both cases, the amount of mannose released was detected by incubation with 250 uL of “developing solution” containing glucose oxidase (55 U/mL), horseradish peroxidase(1 purpurogallin unit/mL), and o dianisidine dihydrochloride (70 ug/mL) for 3 hours and 37 C. The final color intensity was determined by measuring the absorbance at 450 nm on a Bio-Tek (Winooski, Vt.) microtiter plate reader. Free mannose was used as a standard. One unit of mannosidase activity is defined as the amount of enzyme that releases I nmol of mannose in 1 minute at 36C.

### Example 9

Alpha-1,2-Mannosidase activity assays. Herscovics, and Jelinek-Kelly, Anal. Biochem. 166: 85-89,1987, describe an assay that can be used to test mannosidase activity from cell lysates. This assay can be used to test the activity of mannosidases of the present disclosure. Briefly, lysates from Sf9 cells(Acc. # CRL-1711) were used for assays of a 1,2-mannosidase activity with [3H] Man9GlcNAc as described previously (Herscovics, and Jelinek-Kelly, Anal. Biochem. 166: 85-89,1987) with some modifications. The cells were seeded into 25-cm2 tissue culture flasks (Corning Glass Works, Corning, N.Y.) at a density of 3 million cells per flask and infected at a multiplicity of 10 plaque forming units (PFU) per cell with either a recombinant baculovirus encoding the Sf9 a-1,2-mannosidasec DNA under the control of the polyhedrin promoter, or wild-type baculovirus as a control. The cells were harvested by centrifugation at selected times after infection, washed with 100 mM Na+-MES (pH 6.0), and lysed using 1 ptL per 30,000 cells of the same buffer containing 0.5% Triton X-100 with or without 80 mM EDTA. Five microliters of the lysates were used in each reaction of the a-1,2-mannosidase activity assays. Cell lysates for negative control reactions were carried out in a total volume of 40 uL containing 75 mMNa+-MES (pH 6.0), 0.0625% Triton X-100, and 6000 c. p. m. of [3H] Man9GlcNAc. In addition, some reactions contained CaCl2, MgCl2, EDTA.

The reactions were incubated at 37 C for 2.5 hours, boiled for 2 minutes, and 250 wL of a 5 mM solution of CaCl2, MgCl2, and MnCl2, and 175 pL of a 2.25 pg/IlL solution of concanavalin A (Boehringer-Mannheim, Indianapolis, Ind.) in 3.8 MNaCl were added to each reaction. The solutions were vortexed gently and incubated at room temperature for two minutes. Then, 1 mL of 25% PEG-8000 was added and the solutions were vortexed and incubated for another 5 minutes at room temperature. The solutions were centrifuged for 2 minutes at 10,000×g, 1 mL of the supernatant was added to 4 mL of a scintillation cocktail (Ultima Gold; Packard Instrument Company, Meriden, Conn.), and radioactivity was measured using a liquid scintillation counter (model LS60001cl Beckman Instruments, Inc., Fullerton, Calif.). (Herscovics and Jelinek-Kelly, Anal. Biochem. 166: 85-89,1987).

Strains, Media and Growth Conditions: Aspergillus nidulans spore color mutant SM222 was grown in CYM liquid medium (10 g glucose; 2 g bactopeptone; 1.5 g casamino acids; 1 g yeast extract; 10 mLIOOX salt solution, 1 mL1000× trace elements, 10 mL 100× vitamin solution, and 10 mL 100× adenine solution per litre).

Stock solutions (IOOX salt, 100× vitamin, 1000× trace elements, 100× adenine) were described in Kalsner et al., Glycoconjugate J. 12: 360-370,1995. Strains were maintained on CYM agar, and spore suspensions were obtained by washing cultured CYM agar plates with 8 mL 0. Tween 80. Mycelia for DNA isolations were obtained by inoculating 500 mL liquid CYM agar with 108 spores, and incubating 24 hours at 30 C with constant agitation (200 rpm). Oligonucleotide Primer Design: The forward primer MANFOR2B was designed by reverse translation of the protein sequence GGLGESFYEY (SEQ ID NO: 13), and the reverse primer MANREV3B was designed from the complement of the reverse translation of the sequence FXLAETLKYLY (SEQ ID NO: 14).

These protein sequences were conserved between the α-1, 2-mannosidase protein sequences of S. cerevisiae (Camirand et al., J. Biol. Chem., 266: 15120-15127, 1991), Mus musculus 1A (Lal et al., J Biol. Chem., 269: 9872-9881,1994; Acc.# U04299), Homo sapiens 1A (Bause et al., Eur. J. Biochem., 217: 535-540,1993; Acc.# X74837), and rabbit liver (Lal et al., J. Biol. Chem., 269: 9872-9881,1994). A codon usage table compiled for A. nidulans was used to aid in nucleotide selection at degenerate sites.

DNA Isolation and PCR Amplification: Total genomic DNA was extracted from finely ground freeze-dried mycelia of A. nidulans strain SM222. Approximately 400 mg of mycelia were vortexed with 2.5 mL of 50 mM EDTA, 0.2% SDS, and centrifuged for 10 minutes. Then, 85 uL of 3 M KOAc, 5 M acetic acid were added to the supernatant. Following a 20-minute incubation on ice, the suspension was re-centrifuged and DNA was isopropanol-precipitated from the supernatant. After resuspension in 100 pL TE (10 mM Tris pH 7.5,1 mM EDTA), the DNA was extracted once with phenol, twice with chloroform/isoamyl alcohol (24: 1) and ethanol precipitated. Each PCR reaction consisted of 10-100 ng of genomic DNA, 50 pmol of each primer, 10 mMTris-HCl (pH 8.3), 1.5 mM MgCl, 50 mMKCI, 0. gelatin, 0. Triton X-100,200M each of dATP, dCTP, dTTP, and dGTP, and 2 units Taq DNA polymerase (Perkin-Elmer, Norwalk, Conn.) in a final volume of 100u. L. Amplification was performed in two stages using a Perkin-Elmer thermal cycler. Five cycles at a lower stringency (56° C.) were followed by 30 cycles at higher stringency (63° C.). The PCR products were eluted from 1% low-melting-point agarose, cloned into T-Vector (Promega, Madison, Wis.) using the T-Vector cloning system and sequenced with

Universal Forward and M 13 Reverse primers (Amersham International, Buckinghamshire, England).

Library Construction and Screening: The cloned PCR products were used as a template to produce a radiolabeled PCR probe for library screening. Briefly, the PCR reactions were performed as previously described, except that only 100 uM dCTP was used and 0.825 uLa-32P-dCTP (5pCi) was added to the reactions (reduction of the dCTP concentration prior to addition of the radiolabeled nuclide reduces error due to base misincorporation). The radiolabeled PCR products were purified using the WIZARD™ PCR Prep Kit (Promega) and scintillation counted to assess radioactivity.

A genomic library of A. nidulans (SM222) sequences was constructed by digesting genomic DNA with BamHI and ligating the resulting fragments into the similarly digested lambda DNA vector EMPL-3. Concatemers of the ligated DNA were packaged using the GIGAPACK II (Stratagene, La Jolla, Calif.) in vitro packaging system. Approximately 105 recombinant lambda plaques were immobilised on nylon membranes (GENESCREENPLUS™, Dupont, Wilmington, Del.) and hybridized with the radiolabeled PCR product generated from A. nidulans. Single hybridizing clones were selected and rescreened. Lambda DNA was isolated with the WIZARD™ Lambda DNA Extraction Kit (Promega), digested with restriction enzymes, and subcloned into the pUC 18 cloning vector.

DNA Preparation and Sequencing of A. nidulans α-1, 2-mannosidase 1A:

Subclones, from the A. nidulans library, that contained the full-length α-1,2 mannosidase genes were identified by Southern analysis and sequenced. Initial manual sequencing of the α-mannosidase 1A gene was performed according to the dideoxynucleotide method using the T7 sequencing kit (Pharmacia). Initial sequence data were obtained with the universal priming sites, and with specific sequence primers (primer walking). Final sequence data were provided by subcloning the fragments using various restriction enzymes and sequencing with an ABI373 automated fluorescent sequencer (Applied Biosystems, Foster, California).

Open reading frames were identified and aligned with known α-1,2-mannosidase sequences. The α-mannosidase 1B gene was sequenced by cloning restriction fragments of the positive lambda subclone into BLUESCRIPT II™ cloning vector (Stratagene, La Jolla Calif.) and sequencing on an ABI373 sequencer, using the universal priming sites of the vector.

Identification of Introns: Total RNA was extracted from fresh mycelia grown in 150 mL liquid media using the guanidine isothiocyanate method (TRIzol, Molecular Research, Cincinnati, Ohio). Approximately 1 gram of fresh mycelia harvested by suction filtration was homogenized with 6 mL of TRlzol reagent, and incubated at 30° C. for 10 minutes. To the homegenate, 1.2 mL chloroform was added, and the mixture was shaken vigorously for 15 seconds, incubated at 30° C. for 2 minutes and centrifuged at 3000 rpm. The upper aqueous phase was removed and 3 mL isopropanol were added to precipitate the RNA. After incubation at 30° C. for 10 minutes, the RNA was pelleted by centrifugation and washed with 70% ethanol/diethylpyrocarbonate (DEPC). The RNA pellet was resuspended in 30 uL H20/DEPC and 5 uL were used for cDNA preparation and PCR amplification.

Reverse-transcriptase PCR was performed using SUPERSCRIPT II™ reverse transcriptase (Gibco BRL) for first-strand cDNA synthesis using an oligo-dT primer followed by PCR amplification of the cDNA. The oligo-dT primer was annealed to the RNA by adding 1 L of oligo-dT primer (500 pg/mL) to 5 uL of RNA (approx. 1-3 ug) and 6 aL of H20/DEPC. The mixture was heated to 70° C. for 10 minutes and then quickly chilled on ice. Reverse transcription was achieved by addition of 4 pL “First Strand Buffer” (250 mM Tris-HCl pH 8.3,375 mM KCI, 15 mM MgCl2), 2 pL 0.1 M dithiothrectol (DTT), 1 pL 10 mM dNTPs, and 1 nL (200 U) SUPERSCRIPT II™ reverse transcriptase, followed by incubation at 42° C. for 50 minutes. After first-strand cDNA synthesis, the RNA was digested by the addition of 1 uL (2 U) RNAase H, and incubated at 37 C for 20 minutes. The cDNA was used to amplify regions containing putative introns for comparison with genomic DNA amplification.

Sequence Comparison: The Class-1 α-mannosidase protein sequences used in the sequence comparisons were obtained from GenBank as follows:

Saccharomyces cerevisiae (Camirand et al., J. Biol. Chem., 266: 15120-15127,1991; Acc.# M63598), Aspergillus satoi (Inoue et al., Biochim. Biophys. Acta, 1253: 141-145,1995; Acc.# D49827), Penicillium citrinum (Yoshida and Ichishima, Biochim. Biophys. Acta, 1263: 159-162,1995; Acc.# D45839), Ophiostoma novo-ulmi (C. J. Eades and W. E. Hintz, unpublished; Acc.# AF129495), Drosophila melanogaster (Kerscher et al., Dev. Biol., 168: 613-626,1995; Acc.# X82640), Spodoptera frugiperda (Kawar et al., Glycobiol. 7: 433-443,1997; Acc.# AF005035), Homo sapiens 1A (Bause et al., Eur. J Biochem., 217: 535-540,1993; Acc.# X74837), Homo sapiens 1B (Tremblay et al., Glycobiol. 8: 585-595,1998; Acc.# AF027156),

Mus musculus IA (Lal et al., J. Biol. Chem. 269: 9872-9881,1994; Acc.# U04299), Mus musculus 1B (Herscovics et al., J. Biol. Chem., 269 9864-9871,9864-9871,1994 Ace.Acc. # U03457), and Sus scrofa (Bieberich et al., Eur. J. Biochem., 246: 681-689,1997; Acc.# Y12503). These sequences were aligned using the ClustalW algorithm (Thompson et al., Nuc. Acid. R. 22: 4673-4680,1994) included in the DNAStar computer package (DNAStar, Madison Wis.). The sequence similarity matrix and dendrogram were also generated with DNAStar.

### Example 10

We expressed recombinant forms of two different Class I α-1,2-mannosidases from A. nidulans. Partial purification and biochemical characterization of the two enzymes revealed that there is functional overlap between the α-1,2-mannoisdases in A. nidulans.

Of the three different Class I α-1,2-mannosidase genes recently identified and cloned from A. nidulans (Eades and Hintz 2000, Gene, 255: 25-34.), the A. nidulans mnsIB was highly similar to the A. satoi and P. citrinum Class I α-1,2-mannosidases and likely represents the homologue, while the mnsIA and mnsIC genes were more distantly related. These latter two genes likely arose from gene duplication events. To determine whether these enzymes exhibited overlapping functions and to compare their activities with α-1,2-mannosidases from other filamentous fungi, we expressed and characterized the α-1,2-mannosidase IB and IC from A. nidulans. This was done in the native Aspergillus expression system, and the protein products were modified such that they were secreted into the extracellular media. In order to analyze the activity and specificity of the α-1,2-mannosidases from A. nidulans, it was necessary to purify significant amounts of the proteins. An advantage of utilizing the native (homologous) Aspergillus expression system is that homologous protein expression is often much more efficient than heterologous protein expression. An additional advantage of this expression system is that there is less chance of unwanted changes to the protein product, thus our subsequent biochemical analyses would be more accurate.

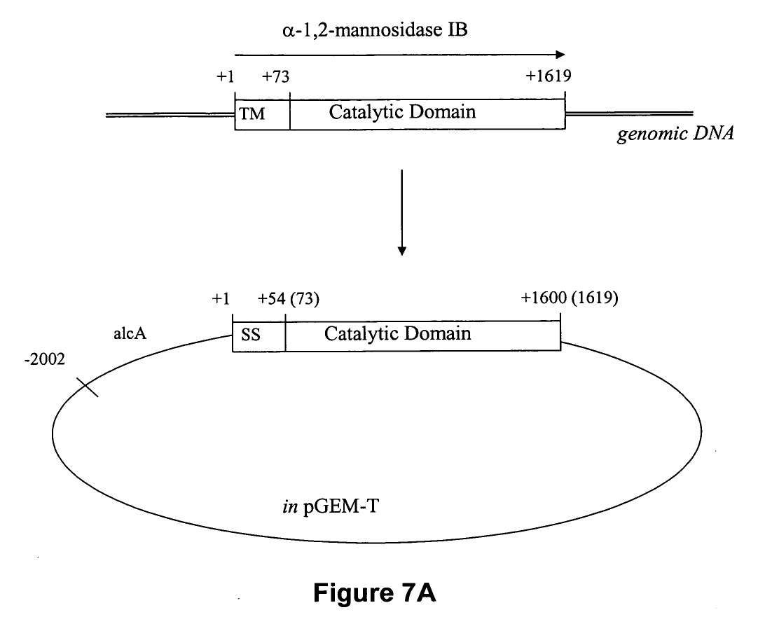

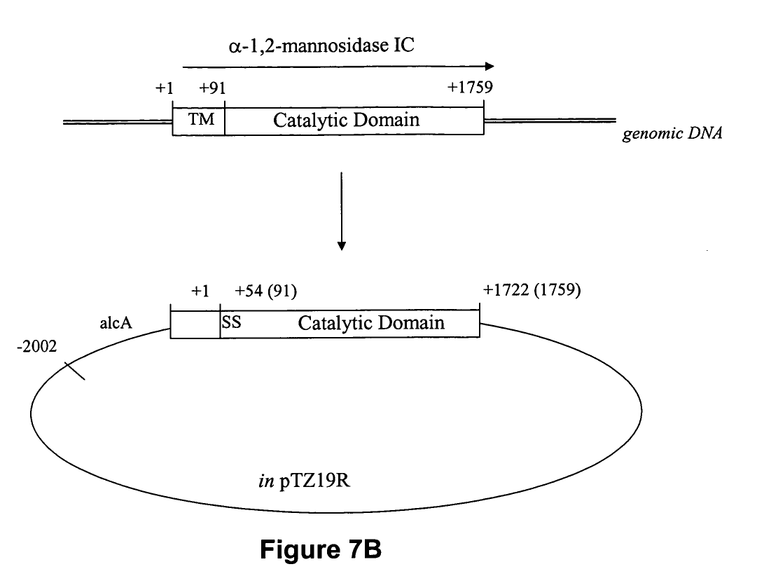

To avoid the difficulty of purifying an intracellular membrane bound enzyme, we directed secretion of the A. nidulans α-1,2-mannosidase IB and IC enzymes to the extracellular media by replacement of the N-terminal transmembrane domains of the proteins with the synthetic secretion signal MDRFLGRHLGLLRHCLRQ (FIG. 7; SEQ ID NO: 20). We chose these two enzymes, since they are the most highly related to each other, and to other known fungal α-1,2-mannosidases. Since the N-terminal regions of the Class I α-1,2-mannosidases are not necessary for the catalytic activity of these proteins (Moremen et al. 1994), removal of this region would not be expected to affect the biochemical properties of the secreted protein.

The α-1,2-mannosidase IB expression vector was created by replacing the N-terminal type-II transmembrane region of the A. nidulans α-1,2-mannosidase IB gene (mnsIB) with a synthetic signal sequence (MDRFLAVISAFFATAFAK; SEQ ID NO: 21) by tailed PCR amplification and fusion to the inducible alcA promoter. The forward primer 5-GGGAAACCATGGACCGGTTCCTCGCCGTCATCTCGGCCTTCTTCGCCACTGCCT TCGCCAAGCGCCCGGTGATGCGCAGTG-3′ (SEQ ID NO: 22) contained an NcoI restriction enzyme site (italics) to facilitate fusion with the alcA promoter. The 5′-overhanging tail is underlined, and the region annealing to the DNA template is marked in bold. The forward primer was designed to anneal at position +91 of the coding region of the α-1,2-mannosidase IB gene, immediately downstream of the transmembrane domain of the original protein. The reverse primer 5′-CACCTCGGCGAGGTTCTTGCG-3′ (SEQ ID NO: 23) was designed to anneal to the reverse complement of the α-1,2-mannosidase IB coding region, from position +471 to +452, immediately downstream of an XbaI restriction enzyme site that was used for cloning. The secretion signal replacement fragment was produced by PCR amplification using high fidelity Pfu Turbo polymerase (Stratagene, La Jolla Calif.). The proofreading capability of the Pfu Turbo polymerase reduced the incidence of base mis-incorporation, however the enzyme also had a tendency to cleave the overhanging regions of the tailed PCR primers. To prevent tail cleavage and provide an extended substrate for subsequent amplification reactions, 5 amplification cycles were initially performed using Taq polymerase (Pharmacia) with the following reaction conditions. Each reaction contained 10-100 ng of genomic DNA, 50 pmol of each primer, 2 μl 10×Taq Buffer (provided), 200 μM each of dATP, dCTP, dTTP, and dGTP and 2 units Taq DNA polymerase in a final volume of 20 μl. Preamplification with Taq polymerase resulted in the creation of sufficient ‘tailed’ template, such that the subsequent amplification using PfuTurbo would not result in tail cleavage. For the second stage of the amplification with PfuTurbo, 1 μl of the Taq PCR reactions as a template for the new reactions. Each reaction contained 10-100 ng of genomic DNA, 50 pmol of each primer, 2 μl 10×Taq Buffer (provided), 200 μM each of dATP, dCTP, dTTP, and dGTP and 2 units Taq DNA polymerase in a final volume of 20 μl. Amplification products were separated by gel electrophoresis on 1.5% agarose gels, and eluted from the gel into 20 μl dH2O. To facilitate cloning into T-Vector (Promega), single A overhangs were added to the eluted DNA by combining 0.5 units Taq polymerase, 50 μM dATP, and 1 μl Taq buffer with the eluted DNA and heating to 72° C. for 10 minutes. By amplifying the tailed fragment by PCR, using the cloned α-1,2-mannosidase IB gene as a template, a fragment of 466 base pairs was obtained. The amplification product was cloned into pGEM-T vector (Promega) and sequenced with an ABI373,automated fluorescent sequencer (Applied Biosystems, Foster City, Calif.) to ensure accuracy. The cloned amplification product, containing the signal sequence and a fragment of the ANIB gene, was then digested by HindIII and XbaI and ligated into similarly digested pUC18 vector. A 2002 bp HindIII/NcoI fragment of the alcA promoter was then ligated into this vector, thus fusing the promoter upstream of the partial coding region. Now that the alcA promoter, signal sequence, and part of the mannosidase IB gene was enclosed in a vector, the remaining portion of the coding region had to be inserted. An XbaI/EcoRI fragment containing the remainder of the mannosidase IB gene was ligated into this vector. The XbaI restriction site utilized for this purpose was the same as the XbaI site in the upstream coding fragment, thus the ligation of the upstream and downstream coding fragments conserved the reading frame and amino acid sequence of the original gene, creating the final expression vector, ANIBSEC (FIG. 7).

The α-1,2-mannosidase IC expression vector was created by replacing the N-terminal type-II transmembrane region of the A. nidulans α-1,2-mannosidase IC (mnsIC) gene with the synthetic signal sequence, MDRFLGRHLGLLRHCLRQ by tailed PCR amplification and fusion to the inducible alcA promoter. The forward primer 5′-GGGAAACCATGGACCGGTTCCTCGCCGTCATCTCGGCCTTCTTCGCCACTGCCT TCGCCAAGACACCACGCGCCGCCAATAGC-3′ (SEQ ID NO: 24) contained an NcoI restriction enzyme site (italics) to facilitate fusion with the alcA promoter. The 5′-overhanging tail is underlined, and the region annealing to the DNA template is marked in bold. The forward primer was designed to anneal at position +91 of the coding region of the mnsIC gene, immediately downstream of the transmembrane domain of the original protein. The reverse primer 5′-GGAGGATGGGGACGAGTGCGG-3′ (SEQ ID NO: 25) was designed to anneal to the reverse complement of the mnsIC coding region, from position +567 to +547, immediately downstream of an EcoRI restriction enzyme site that was used for cloning. The tailed PCR products were ligated into pGEM-T (Promega) and ligations were electroporated into E. coli DH10B (Stratagene, La Jolla, Calif.). Positive clones identified by digestion with NcoI and EcoRI were sequenced. The NcoI/EcoRI fragment from a clone containing the correct DNA sequence was inserted into the similarly digested pGUET vector. This vector contains the alcA promoter in the cloning vector pTZ19R, with an NcoI site at the 3′ end of the promoter. This resulted in the direct fusion of the alcA promoter to the secretion signal linker of the mnsIC gene. The alcA-secretion linker was then moved to BlueScriptII (Stratagene) cloning vector using the flanking HindIII and EcoRI sites. The remainder of the mnsIC gene was then added to the alcA-secretion linker by first inserting the EcoRI/BamHI fragment (the 5′ end of the gene) of the mnsIC coding region. Finally, the remainder of the mnsIC gene and 3′-flanking region was added by inserting the BamHI/SacII fragment from the 3′ end of the gene to create the final vector ANICSEC (FIG. 7).

The α-mannosidase IB and IC expression vectors were introduced into the genome of A. nidulans strain T580 (ura-) by cotransformation and complementation with the pJR15 vector, which contained the selectable ura marker. It has been shown that when two plasmids are used simultaneously in the transformation of fungal protoplasts (cotransformation), as many as 60% of transformed protoplasts will integrate both of the plasmids into the genome (Wernars et al. 1987, Mol. Gen. Genet., 209: 71-77). Protoplasts were prepared according to the method of Fincham (1989, Microbiol. Rev., 53: 148-170), using Sigma Lysing Enzyme for cell wall digestion. Protoplasts of strain T580 (uridine auxotroph) were co-transformed with 1 μg of the selectable marker pJR15, which converts transformed cells to uridine prototrophy, and with 1 μg of either ANIBSEC or ANICSEC. Transformants were initially screened for integration of pFB94 by selection on minimal media, and positive transformants were then transferred to individual complete media plates. Transformants were selected for prototrophy on uridine deficient minimal media and transferred to complete media plates. To confirm secretion of active recombinant α-1,2-mannosidase IB or IC into the culture media of putative transformants, specific activity assays were performed. After 60-65 hours of incubation, aliquots of culture media were assayed for α-1,2-mannosidase activity and compared to the non-transformed T580 strain. Total culture filtrate protein was precipitated and resuspended in assay buffer. Mannosidase assays were performed using the disaccharide Man-α-1,2-Man-OCH3 as a substrate in a coupled enzyme assay as described earlier (Scaman et al. 1996, Glycobiol., 6: 265-270), with some modifications. Digestion of the substrate was performed in a 30 μl final volume containing 10 mM sodium acetate/acetic acid buffer (pH 5.0) and 3 μl 100 mM disaccharide Man-α-1,2-Man-OCH3 incubated at 37° C. for 3 hours. Released mannose was detected by addition of 30 μl 1.25M Tris-HCl (pH7.6) and 240 μl of developing solution, containing glucose oxidase (55 U/ml), horseradish peroxidase (1 U/ml) and o-dianisidine dihydrochloride (70 μg/ml) followed by incubation at 37° C. for 3 hours. Absorbance measurements at 450 nm were used to determine final color change. A standard curve of free mannose concentrations was determined for comparison. One unit of enzyme activity was defined as the amount of enzyme that released 1 nmol of mannose per hour. Specific α-mannosidase activity was standardized by comparison with total protein in the enzyme extracts, and was defined as the units of enzyme activity per μg of total protein. Protein concentrations were determined by the Bradford method (Bradford 1976, Anal. Biochem., 72: 248-254) and comparison to a bovine serum albumin (BSA) standard curve. Crude assays revealed several transformants with secreted α-1,2-mannosidase activity which was significantly higher than non-transformed strains. Crude extracellular filtrates from non-transformed strains contained little to no endogenous α-1,2-mannosidase activity. The transformants that gave the highest yield of crude enzyme were selected for further purification of these enzymes.

In order to characterize the biochemical properties of these recombinant enzymes, we further purified the expressed enzymes from highly expressing strains. Culture filtrates from strains expressing either α-1,2-mannosidase IB or IC were precipitated with stepwise concentrations of ammonium sulfate. The mannosidase activity was retained in the 40-70% ammonium sulfate fraction. Precipitated protein was resuspended in 10 mM PIPES buffer (pH 6.0) and applied to Superose 6 HR 10/30 gel permeation chromatography column attached to an FPLC instrument (Pharmacia). Proteins were eluted using the same buffer at a flow rate of 0.5 ml/min. Active fractions were pooled and loaded onto a MonoQ HR 5/5 anion exchange column. Proteins were eluted using an increasing NaCl gradient. The flow rate was 1.0 m/min., using a 20 min. gradient from 0 to 1 M NaCl. Active fractions were pooled and buffer exchanged into either 10 mM sodium acetate/acetic acid (pH 5.0) or 10 mM PIPES (pH 6.0).

The α-1,2-mannosidase IB was purified to a specific activity of 7.52 U/μg, using non-limiting amounts of the synthetic substrate Man-α-1,2-Man-OCH3, while the α-1,2-mannosidase IC was purified to a specific activity of 4.56 U/Pg. This is comparable to the specific activity of the purified T. reesei enzyme (Maras et al. 2000, J. Biotechnol., 77: 255-263). The molecular mass of the proteins were determined by MALDI-TOF mass spectrometry. The α-1,2-mannosidase IB had a molecular mass of 60 kD while the α-1,2-mannosidase IC had a molecular mass of 63 kD.

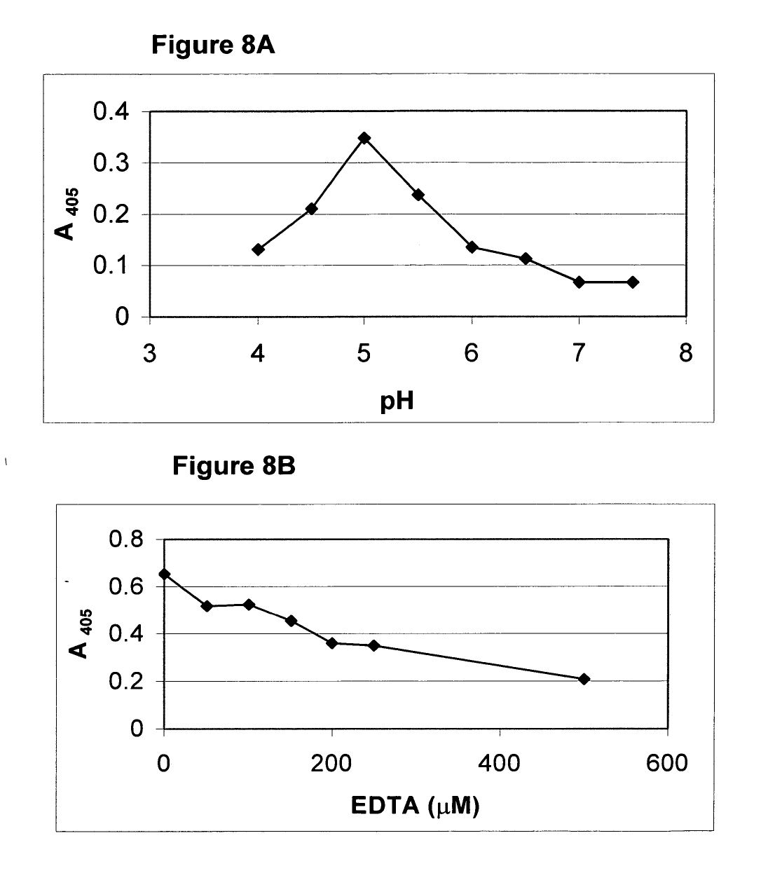

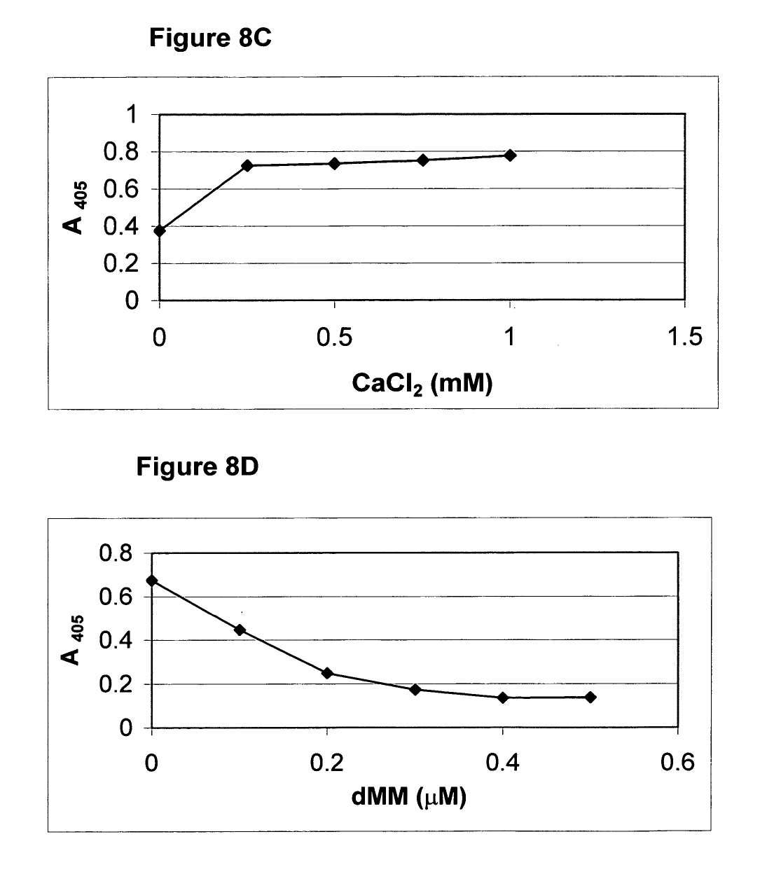

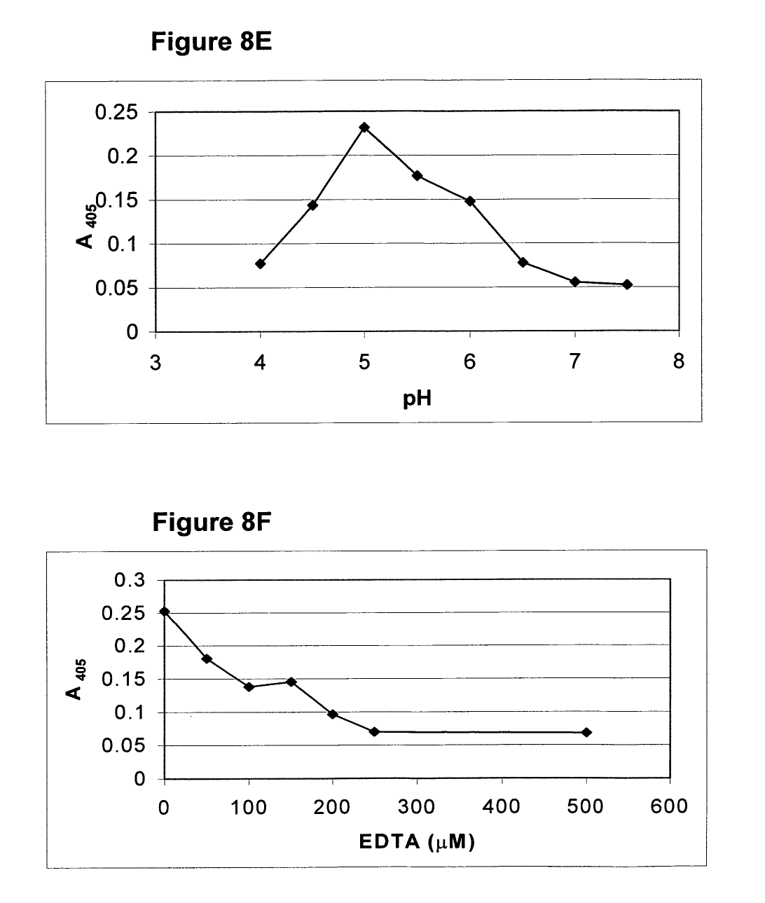

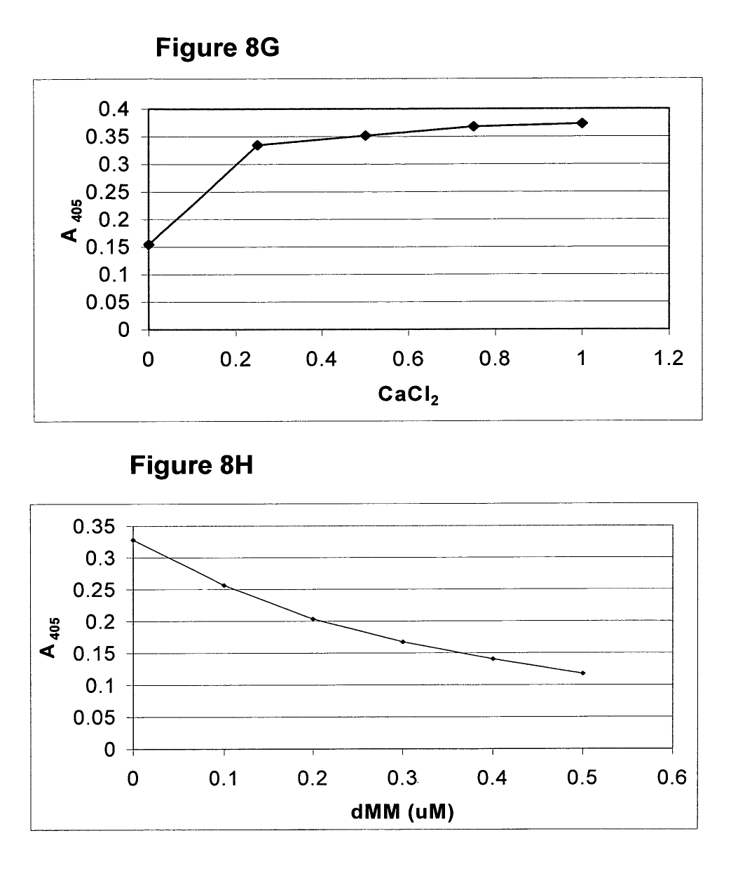

The optimal pH for highest activity for both the α-1,2-mannosidase IB and α-1,2-mannosidase IC was determined to be 5.0 (FIG. 8). Activity dropped considerably more than one pH unit above or below this optimum. The dependence of the activity of these proteins on calcium was verified by the addition of the ion chelator EDTA, which significantly reduced enzyme activity. This activity was restored by the addition of calcium. Addition of the inhibitor deoxymannojirimycin, a potent inhibitor of Class I α-mannosidases, also caused a significant decrease in activity of the α-1,2-mannosidase IB and α-1,2-mannosidase IC enzymes. These data are consistent with the properties of other fungal α-1,2-mannosidases, such as those isolated from T. reesei, A. satoi, and P. citrinum. It should be noted that a second mannosidase, which was partially purified from P. citrinum, and was reported to cleave Man9GlcNAc2 to Man8GlcNAc2, had optimal activity at pH 7.0 (Yoshida et al. 1998, Biosci. Biotechnol. Biochem., 62: 309-315 ).

The Class I α-1,2-mannosidases have been generally classified into three functional subgroups (Lobsanov et al. 2002, J. Biol. Chem., 277: 5620-5630). The first subgroup of enzymes includes the yeast and human α-1,2-mannosidases that reduce Man9GlcNAc2 to Man8GlcNAc2 isomer B. These enzymes are generally found in the ER. The second subgroup includes mammalian Golgi α-1,2-mannosidases, insect α-1,2-mannosidases, and the fungal α-1,2-mannosidases from T. reesei, A. satoi, and P. citrinum. These enzymes primarily cleave Man9GlcNAc2 down to Man5GlcNAc2 through a Man8GlcNAc2 isomer A or isomer C intermediate. A second α-1,2-mannosidase which was partially purified from A. oryzae appeared to cleave Man9GlcNAc2 to Man8GlcNAc2 isomer B, which would indicate that it belongs in the first subgroup of enzymes. The nature of the substrate binding specificity and the cleavage products produced appears to be determined by the shape and size of the substrate binding pocket. This was determined by comparison of the X-ray crystallography structures of P. citrinum and S. cerevisiae structures (Lobsanov et al. 2002, J. Biol. Chem., 277: 5620-5630), and was also demonstrated in the T. reesei crystal structure (Van Petegem et al. 2001, J. Mol. Biol., 312: 157-165). The third subgroup of Class I α-1,2-mannosidases involves an ER mannosidase-like protein which is involved in glycoprotein degradation, and does not hydrolyze Man9GlcNAc2 (Hosokawa et al. 2001, EMBO Reports, 2: 415-422; Nakatsukasa et al. 2001, J. Biol. Chem., 276: 8635-8638).

The substrate specificity of the α-1,2-mannosidase IB and IC from A. nidulans was determined by analyzing the cleavage products of Man9GlcNAc2 by MALDI-TOF mass spectrometry. Approximately 100 ng of either α-1,2-mannosidase IB or IC protein was used to digest 10 pmol of Man9GlcNAc2 (Sigma) in a final volume of 10 μl. Digestion products were analyzed by matrix assisted laser desorption ionization time of flight mass spectrometry (MALDI-TOF-MS) using a Voyager biospectrometry workstation (Applied Biosystems, Foster City Calif.). Samples were mixed in a 1:1 ratio with the matrix (10 mg/ml 2,5-dihydroxybenzoic acid; 50% acetonitrile) and 1 μl was loaded onto a sample plate for analysis. The instrument was set in linear positive mode which increased the sensitivity of the analysis, but also decreased the resolution. Accelerating voltage was 20000V and the grid voltage was set at 94%. Data were analyzed using Data Explorer software (Applied Biosystems, Foster City Calif.). Under non-limiting conditions, we found that both the α-1,2-mannosidase IB and α-1,2-mannosidase IC enzymes cleaved Man9GlcNAc2 completely and efficiently to a Man5GlcNAc2 structure. This supports the classification of both the α-1,2-mannosidase IB and IC in subgroup 2.

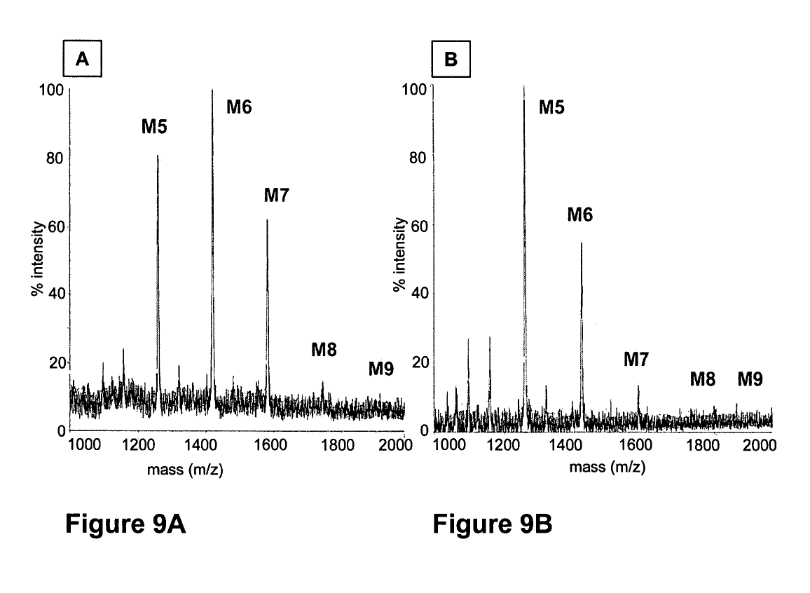

The rate at which various intermediates were produced during the digestion of Man9GlcNAc2 to Man5GlcNAc2 was determined by analyzing the cleavage products during limited time-course experiments (FIG. 9). Both the α-1,2-mannosidase IB and IC enzyme rapidly degraded Man9GlcNAc2 to Man7GlcNAc2, but then proceeded much more slowly to Man6GlcNAc2 and on to Man5GlcNAc2. This led to the accumulation of Man7GlcNAc2 and Man6GlcNAc2 intermediates during the progress of the digestion, which were eventually converted to Man5GlcNAc2. Significantly, the α-1,2-mannosidase IC enzyme converted these intermediates to Man5GlcNAc2 more rapidly than the α-1,2-mannosidase IB enzyme, even though the α-1,2-mannosidase IB had a higher specific activity towards the synthetic substrate Man-α-1,2-Man-OCH3, thus illustrating the significant difference in the substrate specificity of these enzymes, and the preferred utilization of the α-1,2-mannosidase IC enzyme for the modification of N-glycans.

Having illustrated and described the principles of the disclosure in multiple embodiments and examples, it should be apparent to those skilled in the art that the disclosure can be modified in arrangement and detail without departing from such principles. We claim all modifications coming within the spirit and scope of the following claims.

1. An isolated nucleic acid molecule encoding a protein having mannosidase activity, wherein the protein comprises:
(a) an amino acid sequence comprising SEQ ID NO: 18;
(b) an amino acid sequence that differs from SEQ ID NO: 18 by one or more conservative amino acid substitutions; or
(c) an amino acid sequence comprising at least 80% sequence identity to SEQ ID NO: 18.

2. A recombinant nucleic acid molecule, comprising a promoter sequence operably linked to the nucleic acid of claim 1.

3. A cell, transformed with the recombinant nucleic acid molecule of claim 2.

4. The transformed cell of claim 3, wherein the cell is an insect cell, a yeast cell, an algae cell, a bacterial cell, a mammalian cell, or a plant cell.

5. A transgenic fungus, comprising the recombinant nucleic acid of claim 2.

6. A method for producing a macromolecule having an altered glycosylation pattern, comprising allowing the transformed cell of claim 3 to produce the macromolecule.

7. The isolated nucleic acid molecule of claim 1, wherein the nucleic acid molecule comprises at least 80% sequence identity to SEQ ID NO: 17.

8. The isolated nucleic acid molecule of claim 7, wherein the nucleic acid molecule comprises at least 90% sequence identity to SEQ ID NO: 17.

9. The isolated nucleic acid molecule of claim 7, wherein the nucleic acid molecule comprises at least 95% sequence identity to SEQ ID NO: 17.

10. The isolated nucleic acid molecule of claim 7, wherein the nucleic acid molecule comprises SEQ ID NO: 17.

11. The isolated nucleic acid molecule of claim 1, wherein the nucleic acid encodes a protein comprising at least 90% sequence identity to SEQ ID NO: 18.

12. The isolated nucleic acid molecule of claim 1, wherein the nucleic acid encodes a protein comprising SEQ ID NO: 18.
